SEQUENCE LISTING (2 sequences)

SEQ ID NO: 1 - protein, 551 aa, Homo Sapiens
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLPHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 2 - protein, 644 aa, Chlamydia trachomatis
MMRFARFCLLVLTLFPQLAFSAEPLRRQDVRKTVDKLVEHHIDTQQISPYILSRSLEDYVRSFDSHKAYLTQDEVFSHAFSEEATRPLFKQYQEDNFSSFKELDTCIQQSISRAREWRSSWLTDSIRVIQDAMSHTIEKKPSAWASSIEEVKQRQYDLLLSYASIYLEDAAKNRYQGKEHALVKLCIRQIENHENPYIGINDHGYRMSPEEEANSFHVRIIKSIAHSLDAHTAYFSQEEALSMRAQLEKGMCGIGVVLKEDIDGVVVKEVLAGGPADKTGSLRVGDIIYRVNGKNIENTPFPGVLDSLRGSPGSSVTLDIHRQNNDHVIQLRREKILLDSRRVDVSYEPYGNGIIGKITLHSFYEGENQVSSEQDLRKAIRELQEKNLLGLVLDIRENTGGFLSQAIKVSGLFLTNGVVVVSRYADGSVKRYRTISPQKFYDGPLAVLVSKSSASAAEIVAQTLQDYGVALIVGDQQTYGKGTIQHQTITGSNSQEDFFKVTVGRYYSPSGKSTQLEGVKSDIVIPSRYAEDKLGERFLEYALPADQYENVINDNLGDLDINIRPWFQKYYSPHLQKPELVWREMLPQLAHNSQERLEKNKNFEIFVQHLKKTNKQDRSFGSNDLQMEESVNIVKDMILLKSIS

(19) United States
(12) Patent Application Publication
(10) Pub. No.: US 2008/0124338 A1
(43) Pub. Date: May 29, 2008

(54) METHODS FOR TREATMENT OF INFLAMMATORY DISEASE AND CHLAMYDIA INFECTIOUS DISEASE

(75) Inventors: Erguang Li, San Diego, CA (US); Sonya Lad, San Diego, CA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: The Scripps Research Institute, La Jolla, CA (US)

(21) Appl. No.: 11/858,771

(22) Filed: Sep. 20, 2007

Related U.S. Application Data
(60) Provisional application No. 60/846,616, filed on Sep. 22, 2006.

(51) Int. Cl. A61K 39/395; A61K 38/16; A61K 38/48; C12Q 1/37; A61K 31/70

(52) U.S. Cl. 424/141.1; 514/2; 514/44; 424/130.1; 424/94.63; 435/23

(57) ABSTRACT

Methods are provided for treatment of inflammation or inflammatory disease. Methods are further provided for treatment of Chlamydia infection or persistent Chlamydia infection.

### CROSS REFERENCE TO RELATED APPLICATION

This application claims the benefit of U.S. Provisional Application No. 60/846,616, filed Sep. 22, 2006, which is hereby incorporated by reference in its entirety.

### STATEMENT OF GOVERNMENT SUPPORT

This invention was made with Government support of grant numbers K22AI 51534 and RO1 CA098160 from the National Institutes of Health. The Government has certain rights in this invention.

### FIELD

The invention relates generally to methods for treatment of inflammation or inflammatory disease. The invention further relates to methods for treatment of Chlamydia infection or persistent Chlamydia infection.

### BACKGROUND

Microbial pathogens can be classified into two broad categories: those that infect the host accidentally and those that do so for growth. Galan, Annu. Rev. Cell Dev. Biol. 17:53, 2001. The outcome of an infection by “accidental” pathogens is commonly associated with severe host inflammatory responses and is often lethal; whereas obligate intracellular bacterial pathogens such as Chlamydia spp. have developed efficient yet poorly defined mechanisms for immune evasion. Hackstadt, Curr. Opin. Microbiol. 1: 82, 1998. Chlamydiae are obligate intracellular bacterial pathogens that infect a broad range of cell types, including those of the eye and genital tract epithelia. Ocular infection of Chlamydia trachomatis is the leading cause of preventable blindness worldwide and urogenital tract infection remains the most prevalent cause of sexually transmitted diseases in the United States, resulting in pelvic inflammatory disease (PID) and infertility. Centers for Disease Control and Prevention, MMWR Morb. Mortal. Wkly. Rep. 52: 16, 2003; Brunham and Rey-Ladino, Nat. Rev. Immunol 5: 149, 2005; Belland et al., Nat. Rev. Microbiol. 2: 530, 2004; Neznanov et al., J. Biol. Chem. 280: 24153-24158, 2005; Levkau et al., Nature Cell Biology 1: 227-233, 1999; Karin et al., Nature Reviews Immunology 5: 749-759, 2005. Although chlamydial components such as LPS are proinflammatory (Ingalls et al., Infect. Immun. 63: 3125, 1995), the infection generally remains asymptomatic and, consequently, most patients are unaware of an infection. A need exists in the art for improved treatments for Chlamydia infectious disease and associated diseases related to sexually transmitted disease, ocular disease, inflammation, cancer, and atherosclerosis.

### SUMMARY

The present invention relates generally to methods for treatment of inflammation or inflammatory disease, and diseases related to or resulting from inflammation. The invention further relates to methods for treatment of Chlamydia infection or persistent Chlamydia infection, and diseases related to or resulting from Chlamydia infection. A method for treating inflammation or inflammatory disease in a mammalian subject is provided which comprises administering an N-terminal p40 fragment of p65/RelA or an analog thereof to the mammalian subject, wherein the p40 fragment or analog thereof is administered in an amount effective to reduce or eliminate the inflammation or inflammatory disease or to prevent its occurrence or recurrence. The N-terminal p40 fragment of p65/RelA or analog thereof can be a dominant-negative molecule, dominant-negative peptide or dominant-negative peptidomimetic. The p40 fragment or analog thereof can have at least 95% sequence identity to amino acids 1 to 351 of SEQ ID NO:1. In a further aspect, p40 fragment or analog thereof for therapeutic treatment can be a small chemical molecule, monoclonal antibody, polyclonal antibody, peptide, peptidomimetic, or a nucleic acid. The therapeutic nucleic acid can encode the p40 fragment or the analog thereof.

A method for treating inflammation or inflammatory disease in a mammalian subject is provided which comprises administering a Chlamydia protease or an analog thereof to the mammalian subject, wherein the Chlamydia protease or analog thereof is administered in an amount effective to reduce or eliminate the inflammation or inflammatory disease or to prevent its occurrence or recurrence. The Chlamydia protease or analog thereof can have at least 95% sequence identity to SEQ ID NO:2. The Chlamydia protease can be a Chlamydia tail-specific protease, for example, Chlamydia trachomatis tail-specific protease CT441, Chlamydia pneumoniae tail-specific protease Cpn0555, or Chlamydia muridarum tail-specific protease TC0725.

A method for treating persistent Chlamydia infection in a mammalian subject is provided which comprises administering an inhibitor of Chlamydia protease, wherein the inhibitor is administered in an amount effective to reduce or eliminate the persistent Chlamydia infection or to prevent its occurrence or recurrence. The inhibitor can be a small chemical compound, short interfering RNA, dominant-negative molecule, short hairpin RNA, ribozyme, antisense oligonucleotide, antibody, peptide or peptidomimetic. The Chlamydia protease can be a Chlamydia tail-specific protease, for example, Chlamydia trachomatis tail-specific protease CT441, Chlamydia pneumoniae tail-specific protease Cpn0555, or Chlamydia muridarum tail-specific protease TC0725.

A method for treating Chlamydia infection in a mammalian subject is provided which comprises administering a polypeptide fragment of Chlamydia protease to induce an immune response in the mammalian subject wherein the polypeptide fragment is administered in an amount effective to reduce or eliminate the Chlamydia infection or to prevent its occurrence or recurrence. In one aspect, the method further comprises administering an adenovirus vector encoding the polypeptide fragment of Chlamydia protease. The immune response can be a cytotoxic T cell response or a humoral immune response. The Chlamydia protease can be a Chlamydia tail-specific protease, for example, Chlamydia trachomatis tail-specific protease CT441, Chlamydia pneumoniae tail-specific protease Cpn0555, or Chlamydia muridarum tail-specific protease TC0725.

An in vitro method of screening for an inhibitor of Chlamydia protease activity is provided which comprises contacting a cell line with a test compound, and detecting a decrease in Chlamydia protease p65/RelA cleavage to p40, thereby identifying the test compound as an inhibitor of Chlamydia protease activity. The cell line can be a Hela 229 cell line, 293T cell line, or NIH3T3 cell line. In a further aspect the method can comprise detecting a decrease in susceptibility of the cell line to Chlamydia infection. The Chlamydia protease can be a Chlamydia tail-specific protease.

A method for identifying a compound capable of inhibiting Chlamydia infection of a cell is provided which comprises contacting a test compound with a cell-based assay system comprising a cell expressing Chlamydia protease and capable of signaling responsiveness to NF-κB, and detecting an effect of the test compound on NF-κB activation in the presence of TNFα in the cell-based assay system as an increase or a decrease in susceptibility of the cell to Chlamydia infection, effectiveness of the test compound in the assay being indicative of the inhibition of Chlamydia infection of the cell. In one aspect the test compound inhibits Chlamydia protease activity and restores NF-κB activation in the presence of TNFα indicating a decrease in susceptibility of the cell line to Chlamydia infection in the presence of the test compound. The test compound can be a small chemical molecule, interfering RNA, dominant-negative molecule, short hairpin RNA, ribozyme, antisense oligonucleotide, protein inhibitor, monoclonal antibody, polyclonal antibody, peptide, peptidomimetic, or a nucleic acid. The cell based assay system can further comprise 293T cells or NIH3T3 cells The Chlamydia protease can be a Chlamydia tail-specific protease.

### BRIEF DESCRIPTION OF THE DRAWINGS

FIGS. 1A, 1B, 1C, 1D and 1E show Chlamydia trachomatis infection does not induce IκBα degradation, but promotes the cleavage of p65/RelA.

FIGS. 2A, 2B and 2C show the p65 cleavage activity is associated with the chlamydial elementary body (EB).

FIGS. 3A, 3B, 3C and 3D show the identification of the chlamydial protease responsible for the cleavage of p65 protein. The figure shows the expression of chlamydial protease or p40 cleavage product blocks NF-κB activation.

### DETAILED DESCRIPTION

### Overview

The present invention provides methods for treatment of inflammation or inflammatory disease in a mammalian subject. The present invention further provides a method for treating Chlamydia infection or persistent Chlamydia infection in a mammalian subject.

Chlamydia trachomatis infection is the leading cause of sexually-transmitted disease in the United States. A hallmark of chlamydial STD is its asymptomatic nature although inflammatory cellular response and chronic inflammation are among the underlying mechanisms. We show here that Chlamydia sp. has the ability to interfere with the NF-κB pathway of host inflammatory response. We found that Chlamydia infection did not promote IκBα degradation, a prerequisite for NF-κB nuclear translocation/activation, nor induce p65 nuclear redistribution. Instead, it caused p65 cleavage into an N-terminus-derived p40 fragment and a p22 of the C-terminus. The chlamydial protein that selectively cleaved p65 was identified as a tail-specific protease. Importantly, expression of either this protease or the p40 cleavage product could block NF-κB activation. Together, these data suggest that Chlamydia sp. has the ability to convert a regulatory molecule of host inflammatory response to a dominant negative inhibitor of the same pathway potentially to minimize inflammation.

C. trachomatis is the causative agent of trachoma, oculogential disease, infant pneumonia and lymphogranuloma venereum (LGV). C. trachomatis has a limited host range and only infects human epithelial cells (one strain can infect mice). The species is divided into three biovars (biological variants): trachoma, LGV and mouse pneumonitis. The human biovars have been further subdivided in to several serovars (serological variants; equivalent to serotypes) that differ in their major outer membrane proteins and which are associated with different diseases. Serovars A, B, Ba, and C cause trachoma prevalent in Asia and Africa. Serovars D through K cause eye disease, conjunctivitis, sexually transmitted disease, urethritis, cervicitis, and respiratory disease such as infant pneumonia. Serovars LGV1, LGV2, and LGV3 cause lymphogranuloma venerium. C. pneumoniae cause upper respiratory tract infection. C. pneumoniae is also associated with atherosclerosis. C. muridarum, (MoPn) is thought to be a mouse strain.

The Chlamydia tail specific protease is conserved among species of Chlamydophila, Chlamydia, and Parachlamydiaceae, for example: C. abortus (mammals), C. psittaci (birds), C. felis (cats), C. caviae (Guinea pig), C. pecorum (mammals), C. pneumoniae (humans), C. trachomatis (humans), C. suis (swine), C. murdarum (mice, hamsters), P. acanthamoebae.

A method for treating inflammation or inflammatory disease in a mammalian subject is provided which comprises administering to the mammalian subject a Chlamydia tail specific protease to produce a protease cleavage product of p65/RelA subunit of NF-κB, e.g., an N-terminal p40 fragment. The Chlamydia tail specific protease can include, but is not limited to, Chlamydia trachomatis tail-specific protease CT441, Chlamydia pneumoniae tail-specific protease Cpn0555, or Chlamydia muridarum tail-specific protease TC0725, or a homologous protein from other Chlamydia serovars or biovars. Alternatively, a method for treating inflammation or inflammatory disease in a mammalian subject can comprise administering to the mammalian subject a protease cleavage fragment which is an N-terminal p40 fragment of a p65/RelA subunit, an analog of the N-terminal p40 fragment, or a dominant-negative molecule, dominant-negative peptide or dominant-negative peptidomimetic.

A recombinant adenovirus expressing a Chlamydia tail specific protease can be useful as an anti-inflammatory and/or anti-cancer therapeutic agent to treat a mammalian subject. Experiments have demonstrated an association between chronic inflammation in a mammalian subject mediated by NF-κB and development of cancer in the mammalian subject.

A method for treating Chlamydia infection or persistent Chlamydia infection in a mammalian subject is provided which comprises administering to the mammalian subject an inhibitor of Chlamydia protease administered in an amount effective to reduce or eliminate the persistent Chlamydia infection or to prevent its occurrence or recurrence. The Chlamydia protease is a Chlamydia tail specific protease including, but not limited to, Chlamydia trachomatis tail-specific protease CT441, Chlamydia pneumoniae tail-specific protease Cpn0555, or Chlamydia muridarum tail-specific protease TC0725, or a homologous tail specific protease from other Chlamydia serovars or biovars.

It is to be understood that this invention is not limited to particular methods, reagents, compounds, compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a cell” includes a combination of two or more cells, and the like.

The term “about” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

A dominant negative polypeptide or peptidomimetic composition can be a dominant-negative mutant within the scope of the invention if a polypeptide or peptidomimetic can block NF-κB activation and reduce or eliminate inflammation or inflammatory disease or prevent its occurrence or recurrence in a mammalian subject. For example, an N-terminal p40 fragment of p65/RelA or an analog or peptidomimetic thereof can block NF-κB activation. A dominant negative polypeptide or peptidomimetic composition can reduce inflammatory disease or chronic inflammation mediated by NF-κB activation and can be useful to treat cancer associated with inflammatory disease or chronic inflammation.

A protease or Chlamydia protease refers to a protease that cleaves the p65/RelA subunit of NF-κB to p40 and p20 fragments, for example, a Chlamydia tail-specific protease. Inactivation of NF-κB reduces inflammation or an inflammatory response, and specifically reduces an inflammatory response to Chlamydia infection. Epitopes of Chlamydia protease protein can be used as a therapeutic treatment to develop a cell mediated immune response to Chlamydia infection. Successful treatment of Chlamydia infection can be used to treat ocular disease, urogenital infections, trachoma, conjunctivitis, pneumonia and lymphogranuloma venereum (LGV), caused by C. trachomatis; bronchitis, sinusitis, pneumonia and possibly atherosclerosis, caused by C. pneumoniae; or pneumonia (psittacosis), caused by C. psittaci.

“Cell-mediated immunity” and “cell-mediated immune response” refer to the immunological defense provided by lymphocytes, such as that defense provided by T cell lymphocytes when they come into close proximity to target cells, for example, a cell mediated immune response to a Chlamydia protease e.g., tail specific protease CT441 or tail-specific protease Cpn0555. A cell-mediated immune response also comprises lymphocyte proliferation, recruitment, invasion, and activation. When “lymphocyte proliferation” is measured, the ability of lymphocytes to proliferate in response to specific antigen is measured. Lymphocyte proliferation is meant to refer to B cell, T-helper cell or cytotoxic T-lymphocyte (CTL) cell proliferation.

“CTL response” refers to the ability of an antigen-responsive T-cell to lyse and kill a cell expressing the specific antigen, e.g., a polypeptide fragment of Chlamydia protease e.g., tail specific protease CT441 or tail-specific protease Cpn0555. Standard, art-recognized CTL assays are performed to measure CTL activity.

“T lymphocyte response” and “T lymphocyte activity” are used here interchangeably to refer to the component of immune response dependent on T lymphocytes (i.e., the proliferation and/or differentiation of T lymphocytes into helper, cytotoxic killer, or suppressor T lymphocytes, the provision of signals by helper T lymphocytes to B lymphocytes that cause or prevent antibody production, the killing of specific target cells by cytotoxic T lymphocytes, and the release of soluble factors such as cytokines that modulate the function of other immune cells).

“Immune response” refers to the concerted action of lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues. “Immune cell response” refers to the response of immune system cells to external or internal stimuli (e.g., antigen, cytokines, chemokines, and other cells) producing biochemical changes in the immune cells that result in immune cell migration, killing of target cells, phagocytosis, production of antibodies, other soluble effectors of the immune response, and the like.

An “effective amount of an antigenic compound” refers to an amount of antigenic compound which, in optional combination with an adjuvant, will cause the subject to produce a specific immunological response to the antigenic compound.

“Epitope” refers to the site on an antigen, e.g., Chlamydia protease, that is recognized and bound by a particular antibody or T-cell receptor. The minimal size of a protein epitope, as defined herein, is about five amino acids, and a protein epitope typically comprises at least eight amino acids. It is to be noted, however, that an epitope might comprise a portion of an antigen other than the amino acid sequence, e.g., a carbohydrate moiety or a lipid moiety. Furthermore, an epitope may be discontinuous, i.e., it comprises amino acid residues that are not adjacent in the polypeptide but are brought together into an epitope by way of the secondary, tertiary, or quaternary structure of the protein.

“Humoral immunity” or “humoral immune response” refers to the form of immunity mediated by antibody molecules secreted in response to immunogenic stimulation, as well as B cell recruitment of cellular and innate responses.

“Immune response” refers to any response to an immunogenic compound by the immune system of a vertebrate subject. Exemplary immune responses include, but not limited to cellular as well as local and systemic humoral immunity, such as CTL responses, including antigen-specific induction of CD8⁺ CTLs, helper T-cell responses, including T-cell proliferative responses and cytokine release, and B-cell responses including antibody response.

“Inducing an immune response” refers to administration of an immunogenic compound or a nucleic acid encoding the immunogenic compound, wherein an immune response is effected, i.e., stimulated, initiated or induced.

“Potentiating an immune response” refers to administration of an immunogenic compound or a nucleic acid encoding the antigenic compound, wherein a preexisting immune response is improved, furthered, supplemented, amplified, increased or prolonged. The immunogenic compound may be, for example, a composition comprising an antigen or hapten associated or conjugated to a carrier, with or without an adjuvant.

“Immunogenic amount” refers to an amount of a compound sufficient to stimulate an immune response, when administered according to the invention. The amount of a compound necessary to provide an immunogenic amount is readily determined by one of ordinary skill in the art, e.g., by preparing a series of compositions of the invention with varying concentrations of antigenic compound, administering such compositions to suitable laboratory animals (e.g., guinea pigs), and assaying the resulting immune response by measuring serum antibody titer, antigen-induced swelling in the skin, and the like.

### Chlamydia Protease Inhibitors

The present invention provides a method for preventing or treating Chlamydia infectious disease in a mammalian subject comprises administering to the mammalian subject a compound capable of inhibiting a Chlamydia protease, for example, a Chlamydia tail-specific protease, wherein the compound is administered in an amount effective to reduce or eliminate the Chlamydia infectious disease or to prevent its occurrence or recurrence. The present invention further provides a method for identifying a compound capable of inhibiting Chlamydia infection of a cell comprising contacting a test compound with a cell-based assay system comprising a cell expressing Chlamydia protease and capable of signaling responsiveness to NF-κB, and detecting an effect of the test compound on NF-κB signaling in the assay system as an increase or a decrease in susceptibility of the cell line to Chlamydia infection, effectiveness of the test compound in the assay being indicative of the inhibition of Chlamydia infection of the cell.

“Susceptibility to Chlamydia infection” refers to susceptibility to an infectious Chlamydia bacteria. Susceptibility to infection with Chlamydia was measured as described herein using an in vitro cell assay incorporating Hela 229 cells, 293T cells or NIH3T3 cells.

“Patient”, “subject” or “mammal” are used interchangeably and refer to mammals such as human patients and non-human primates, as well as experimental animals such as rabbits, rats, and mice, and other animals. Animals include all vertebrates, e.g., mammals and non-mammals, such as sheep, dogs, cows, chickens, amphibians, and reptiles.

“Treating” or “treatment” includes the administration of the compositions, compounds or agents of the present invention to prevent or delay the onset of the symptoms, complications, or biochemical indicia of a disease, alleviating or ameliorating the symptoms or arresting or inhibiting further development of the disease, condition, or disorder (e.g., a Chlamydia infectious disease). “Treating” further refers to any indicia of success in the treatment or amelioration or prevention of the disease, condition, or disorder (e.g., a Chlamydia infectious disease), including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with a Chlamydia infectious disease. The term “therapeutic effect” refers to the reduction, elimination, or prevention of the disease, symptoms of the disease, or side effects of the disease in the subject. “Treating” or “treatment” using the methods of the present invention includes preventing the onset of symptoms in a subject that can be at increased risk of a Chlamydia infectious disease but does not yet experience or exhibit symptoms, inhibiting the symptoms of a Chlamydia infectious disease (slowing or arresting its development), providing relief from the symptoms or side-effects of Chlamydia infectious disease (including palliative treatment), and relieving the symptoms of Chlamydia infectious disease (causing regression). Treatment can be prophylactic (to prevent or delay the onset of the disease, or to prevent the manifestation of clinical or subclinical symptoms thereof) or therapeutic suppression or alleviation of symptoms after the manifestation of the disease or condition.

The ability of a molecule to bind to Chlamydia protease can be determined, for example, by the ability of the putative ligand to bind to Chlamydia protease immunoadhesin coated on an assay plate. Specificity of binding can be determined by comparing binding to non-Chlamydia protease immunoadhesin.

In one embodiment, antibody binding to Chlamydia protease can be assayed by either immobilizing the ligand or the receptor. For example, the assay can include immobilizing Chlamydia protease fused to a His tag onto Ni-activated NTA resin beads. Antibody can be added in an appropriate buffer and the beads incubated for a period of time at a given temperature. After washes to remove unbound material, the bound protein can be released with, for example, SDS, buffers with a high pH, and the like and analyzed.

“Modulator” includes inhibitors and activators. Inhibitors are agents that, e.g., bind to, partially or totally block stimulation, decrease, prevent, delay activation, inactivate, desensitize, or down regulate the activity Chlamydia protease, e.g., antagonists. Activators are agents that, e.g., bind to, stimulate, increase, open, activate, facilitate, enhance activation, sensitize or up regulate the activity of Chlamydia protease, e.g., agonists. Modulators include agents that, e.g., alter the interaction of Chlamydia protease with proteins that bind activators or inhibitors, receptors, including proteins, peptides, lipids, carbohydrates, polysaccharides, or combinations of the above, e.g., lipoproteins, glycoproteins, and the like. Modulators include genetically modified versions of naturally-occurring Chlamydia protease ligands, e.g., with altered activity, as well as naturally occurring and synthetic ligands, antagonists, agonists, small chemical molecules and the like. Such assays for inhibitors and activators include, e.g., applying putative modulator compounds to a cell expressing a Chlamydia protease and then determining the functional effects on Chlamydia protease activity, as described herein. Samples or assays comprising Chlamydia protease that are treated with a potential activator, inhibitor, or modulator are compared to control samples without the inhibitor, activator, or modulator to examine the extent of inhibition. Control samples (untreated with inhibitors) can be assigned a relative Chlamydia protease is activity value of 100%. Inhibition of Chlamydia protease is achieved when the Chlamydia protease activity value relative to the control is about 80%, optionally 50% or 25-0%. Activation of Chlamydia protease is achieved when the Chlamydia protease activity value relative to the control is 110%, optionally 150%, optionally 200-500%, or 1000-3000% higher.

“Inhibitors,” “activators,” and “modulators” of Chlamydia protease, e.g., tail specific protease CT441 activity or tail-specific protease Cpn0555 activity, are used to refer to inhibitory, activating, or modulating molecules, respectively, identified using in vitro and in vivo assays for Chlamydia protease activity, e.g., ligands, binding partners, agonists, antagonists, and their homologs and mimetics. “Modulator” includes inhibitors and activators. Inhibitors are agents that, e.g., bind to, partially or totally block stimulation, decrease, prevent, delay activation, inactivate, desensitize, or down regulate the activity of Chlamydia protease, e.g., antagonists. Activators are agents that, e.g., bind to, stimulate, increase, open, activate, facilitate, enhance activation, sensitize or up regulate the activity of Chlamydia protease, e.g., agonists. Modulators include agents that, e.g., alter the interaction of Chlamydia protease with proteins that bind activators or inhibitors, receptors, including proteins, peptides, lipids, carbohydrates, polysaccharides, or combinations of the above, e.g., lipoproteins, glycoproteins, and the like. Modulators include genetically modified versions of naturally-occurring Chlamydia protease, e.g., tail specific protease CT441 or tail-specific protease Cpn0555, with altered activity, as well as naturally occurring and synthetic ligands, antagonists, agonists, small chemical molecules and the like. Such assays for inhibitors and activators include, e.g., applying putative modulator compounds to a cell expressing Chlamydia protease, e.g., tail specific protease CT441 or tail-specific protease Cpn0555, and then determining the functional effects on Chlamydia infection in the cell, as described herein. Samples or assays comprising Chlamydia protease, e.g., tail specific protease CT441 or tail-specific protease Cpn0555, are treated with a potential activator, inhibitor, or modulator are compared to control samples without the inhibitor, activator, or modulator to examine the extent of inhibition. Control samples (untreated with inhibitors) can be assigned a relative Chlamydia protease activity value of 100%. Inhibition of Chlamydia infection is achieved when the Chlamydia protease activity value relative to the control is about 80%, optionally 50% or 25-0%.

“Antagonist” is used in the broadest sense, and includes any molecule that partially or fully blocks, inhibits, or neutralizes a biological activity of Chlamydia protease, e.g., tail specific protease CT441 or tail-specific protease Cpn0555, activity. In a similar manner, the term “agonist” is used in the broadest sense and includes any molecule that mimics or enhances a biological activity of Chlamydia protease, e.g., tail specific protease CT441 or tail-specific protease Cpn0555 activity. Suitable agonist or antagonist molecules specifically include agonist or antagonist antibodies or antibody fragments, fragments or amino acid sequence variants of native Chlamydia protease polypeptides, peptides, antisense oligonucleotides, small organic molecules, and the like. Methods for identifying agonists or antagonists of Chlamydia protease polypeptides can comprise contacting an Chlamydia protease polypeptide with a candidate agonist or antagonist molecule and measuring a detectable change in one or more biological activities normally associated with the Chlamydia protease.

“Test compound” refers to a nucleic acid, DNA, RNA, protein, polypeptide, or small chemical entity that is determined to effect an increase or decrease in a gene expression or actin cytoskeleton rearrangement as a result of signaling through Chlamydia protease, e.g., tail specific protease CT441 or tail-specific protease Cpn0555. The test compound can be an antisense RNA, ribozyme, polypeptide, or small molecular chemical entity. The term “test compound” can be any small chemical compound, or a biological entity, such as a protein, sugar, nucleic acid or lipid. Typically, test compounds will be small chemical molecules and polypeptides. A “test compound specific for signaling through Chlamydia protease” is determined to be a modulator of Chlamydia protease activity.

“Cell-based assays” include Chlamydia protease binding assays, for example, radioligand or fluorescent ligand binding assays for Chlamydia protease activity or binding of the protein, e.g., tail specific protease CT441 or tail-specific protease Cpn0555, in cells, plasma membranes, detergent-solubilized plasma membrane proteins, immobilized collagen (Alberdi, J Biol. Chem. 274:31605-12, 1999; Meyer et al., 2002); Chlamydia protease-affinity column chromatography (Alberdi, J Biol Chem. 274:31605-12, 1999; Aymerich et al., Invest Ophthalmol Vis Sci. 42:3287-93, 2001); Chlamydia protease ligand blot using a radio- or fluorosceinated-ligand (Aymerich et al., Invest Ophthalmol Vis Sci. 42:3287-93, 2001; Meyer et al., 2002); Size-exclusion ultrafiltration (Alberdi et al., Biochem., 1998; Meyer et al., 2002); or ELISA. Exemplary Chlamydia protease binding activity assays of the present invention are: a Chlamydia protease ligand blot assay (Aymerich et al., Invest Ophthalmol Vis Sci. 42:3287-93, 2001); a Chlamydia protease affinity column chromatography assay (Alberdi, J Biol. Chem. 274:31605-12, 1999) and a Chlamydia protease ligand binding assay (Alberdi et al., J Biol Chem. 274:31605-12, 1999). Each incorporated by reference in their entirety.

In one embodiment, Chlamydia protease, e.g., tail specific protease CT441 or tail-specific protease Cpn0555, can be assayed by either immobilizing the ligand/interacting protein or the protease. For example, the assay can include immobilizing Chlamydia protease fused to a His tag onto Ni-activated NTA resin beads. Inhibitors of Chlamydia protease can be added in an appropriate buffer and the beads incubated for a period of time at a given temperature. After washes to remove unbound material, the bound protein can be released with, for example, SDS, buffers with a high pH, and the like and analyzed.

“Contacting” refers to mixing a test compound in a soluble form into an assay system, for example, a cell-based assay system, such that an effect upon receptor-mediated signaling can be measured.

“Signaling in cells” refers to the interaction of a ligand with a protease, or interaction of p65/RelA with a protease, such as Chlamydia protease, e.g., tail specific protease CT441 or tail-specific protease Cpn0555, to produce a response, for example, a T cell mediated immune response, or a response to prevent or alleviate Chlamydia infectious disease. “Signaling responsiveness” or “effective to activate signaling” or “stimulating a cell-based assay system” refers to the ability of inhibitors of Chlamydia protease activity to stimulate an immune response, and to prevent or alleviate a Chlamydia infectious disease.

“Detecting an effect” refers to an effect measured in a cell-based assay system. For example, the effect detected can be Chlamydia protease activity, e.g., tail specific protease CT441 or tail-specific protease Cpn0555, in an assay system, for example, a Hela 229 cell, 293T cell or NIH3T3 cell in vitro assay.

“Assay being indicative of modulation” refers to results of a cell-based assay system indicating that cell activation by Chlamydia protease, e.g., tail specific protease CT441 or tail-specific protease Cpn0555, induces a protective response in cells against a Chlamydia infectious disease.

“Biological activity” and “biologically active” with regard to an inhibitor of Chlamydia protease, e.g., tail specific protease CT441 or tail-specific protease Cpn0555, of the present invention refer to the ability of the inhibitor molecule to specifically bind to and signal through a native or recombinant Chlamydia protease, or to block the ability of a native or recombinant Chlamydia protease to participate in signal transduction via NF-κB. Thus, the (native and variant) ligands of Chlamydia protease of the present invention include agonists and antagonists of a native or recombinant Chlamydia protease. Preferred biological activities of the ligands of Chlamydia protease, e.g., tail specific protease CT441 or tail-specific protease Cpn0555, of the present invention include the ability to enhance an immune response, or treat a Chlamydia infectious disease. Accordingly, the administration of the compounds or agents of the present invention can prevent or delay, to alleviate, or to arrest or inhibit development of the symptoms or conditions associated with a Chlamydia infectious disease, or other disorders.

“Concomitant administration” of a known drug with a compound of the present invention means administration of the drug and the compound at such time that both the known drug and the compound will have a therapeutic effect or diagnostic effect. Such concomitant administration can involve concurrent (i.e. at the same time), prior, or subsequent administration of the drug with respect to the administration of a compound of the present invention. A person of ordinary skill in the art, would have no difficulty determining the appropriate timing, sequence and dosages of administration for particular drugs and compounds of the present invention.

In general, the phrase “well tolerated” refers to the absence of adverse changes in health status that occur as a result of the treatment and would affect treatment decisions.

“Lymphocyte” as used herein has the normal meaning in the art, and refers to any of the mononuclear, nonphagocytic leukocytes, found in the blood, lymph, and lymphoid tissues, i.e., B and T lymphocytes.

“Subpopulations of T lymphocytes” or “T cell subset(s)” refers to T lymphocytes or T cells characterized by the expression of particular cell surface markers (see Barclay, A. N. et al., (eds.), THE LEUKOCYTE ANTIGEN FACTS BOOK, 2^{ND}. EDITION, Academic Press, London, United Kingdom, 1997; this reference is herein incorporated by reference for all purposes).

“Epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

An intact “antibody” comprises at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, C_{H}1, C_{H}2 and C_{H}3. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) through cellular receptors such as Fc receptors (e.g., FcγRI, FcγRIIa, FcγRIIb, FcγRIII, and FcRη) and the first component (Clq) of the classical complement system. The term antibody includes antigen-binding portions of an intact antibody that retain capacity to bind the antigen. Examples of antigen binding portions include (i) a Fab fragment, a monovalent fragment consisting of the V_{L}, V_{H}, C_{L} and C_{H}1 domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., Nature 341: 544-546, 1989), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); See, e.g., Bird et al., Science 242: 423-426, 1988; and Huston et al., Proc. Natl. Acad. Sci. U.S.A. 85: 5879-5883, 1988). Such single chain antibodies are included by reference to the term “antibody” Fragments can be prepared by recombinant techniques or enzymatic or chemical cleavage of intact antibodies.

“Human sequence antibody” includes antibodies having variable and constant regions (if present) derived from human immunoglobulin sequences. The human sequence antibodies of the invention can include amino acid residues not encoded by human germline immunoglobulin sequences (e.g., mutations introduced by random or site-specific mutagenesis in vitro or by somatic mutation in vivo). However, the term “human sequence antibody”, as used herein, is not intended to include antibodies in which entire CDR sequences sufficient to confer antigen specificity and derived from the germline of another mammalian species, such as a mouse, have been grafted onto human framework sequences (i.e., humanized antibodies).

“Monoclonal antibody” or “monoclonal antibody composition” refer to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions (if present) derived from human germline immunoglobulin sequences. In one embodiment, the human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

“Diclonal antibody” refers to a preparation of at least two antibodies to an antigen. Typically, the different antibodies bind different epitopes.

“Oligoclonal antibody” refers to a preparation of 3 to 100 different antibodies to an antigen. Typically, the antibodies in such a preparation bind to a range of different epitopes.

“Polyclonal antibody” refers to a preparation of more than 1 (two or more) different antibodies to an antigen. Such a preparation includes antibodies binding to a range of different epitopes.

“Recombinant human antibody” includes all human sequence antibodies of the invention that are prepared, expressed, created or isolated by recombinant means, such as antibodies isolated from an animal (e.g., a mouse) that is transgenic for human immunoglobulin genes (described further below); antibodies expressed using a recombinant expression vector transfected into a host cell, antibodies isolated from a recombinant, combinatorial human antibody library, or antibodies prepared, expressed, created or isolated by any other means that involves splicing of human immunoglobulin gene sequences to other DNA sequences. Such recombinant human antibodies have variable and constant regions (if present) derived from human germline immunoglobulin sequences. Such antibodies can, however, be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

A “heterologous antibody” is defined in relation to the transgenic non-human organism producing such an antibody. This term refers to an antibody having an amino acid sequence or an encoding nucleic acid sequence corresponding to that found in an organism not consisting of the transgenic non-human animal, and generally from a species other than that of the transgenic non-human animal.

A “heterohybrid antibody” refers to an antibody having a light and heavy chains of different organismal origins. For example, an antibody having a human heavy chain associated with a murine light chain is a heterohybrid antibody.

“Substantially pure” or “isolated” means an object species (e.g., an antibody of the invention) has been identified and separated and/or recovered from a component of its natural environment such that the object species is the predominant species present (i.e., on a molar basis it is more abundant than any other individual species in the composition); a “substantially pure” or “isolated” composition also means where the object species comprises at least about 50 percent (on a molar basis) of all macromolecular species present. A substantially pure or isolated composition can also comprise more than about 80 to 90 percent by weight of all macromolecular species present in the composition. An isolated object species (e.g., antibodies of the invention) can also be purified to essential homogeneity (contaminant species cannot be detected in the composition by conventional detection methods) wherein the composition consists essentially of derivatives of a single macromolecular species. For example, an isolated antibody to Chlamydia protease can be substantially free of other antibodies that lack binding to human Chlamydia protease and bind to a different antigen. Further, an isolated antibody that specifically binds to an epitope, isoform or variant of human Chlamydia protease may, however, have cross-reactivity to other related antigens, e.g., from other species (e.g., Chlamydia protease species homologs). Moreover, an isolated antibody of the invention be substantially free of other cellular material (e.g., non-immunoglobulin associated proteins) and/or chemicals.

“Specific binding” refers to preferential binding of an antibody to a specified antigen relative to other non-specified antigens. The phrase “specifically (or selectively) binds” to an antibody refers to a binding reaction that is determinative of the presence of the protein in a heterogeneous population of proteins and other biologics. Typically, the antibody binds with an association constant (Kₐ) of at least about 1×10⁶ M⁻¹ or 10⁷ M⁻¹, or about 10⁸ M⁻¹ to 10⁹ M⁻¹, or about 10¹⁰ M⁻¹ to 10¹¹ M⁻¹ or higher, and binds to the specified antigen with an affinity that is at least two-fold greater than its affinity for binding to a non-specific antigen (e.g., BSA, casein) other than the specified antigen or a closely-related antigen. The phrases “an antibody recognizing an antigen” and “an antibody specific for an antigen” are used interchangeably herein with the term “an antibody which binds specifically to an antigen”. A predetermined antigen is an antigen that is chosen prior to the selection of an antibody that binds to that antigen.

“Specifically bind(s)” or “bind(s) specifically” when referring to a peptide refers to a peptide molecule which has intermediate or high binding affinity, exclusively or predominately, to a target molecule. The phrases “specifically binds to” refers to a binding reaction which is determinative of the presence of a target protein in the presence of a heterogeneous population of proteins and other biologics. Thus, under designated assay conditions, the specified binding moieties bind preferentially to a particular target protein and do not bind in a significant amount to other components present in a test sample. Specific binding to a target protein under such conditions can require a binding moiety that is selected for its specificity for a particular target antigen. A variety of assay formats can be used to select ligands that are specifically reactive with a particular protein. For example, solid-phase ELISA immunoassays, immunoprecipitation, Biacore™ and Western blot are used to identify peptides that specifically react with the antigen. Typically a specific or selective reaction will be at least twice background signal or noise and more typically more than 10 times background.

“High affinity” for an antibody refers to an equilibrium association constant (Kₐ) of at least about 10⁷ M⁻¹, at least about 10⁸ M⁻¹, at least about 10⁹ M⁻¹, at least about 10¹⁰ M⁻¹, at least about 10¹¹ M⁻¹, or at least about 10¹² M⁻¹ or greater, e.g., up to 10¹³ M⁻¹ or 10¹⁴ M⁻¹ or greater. However, “high affinity” binding can vary for other antibody isotypes.

The term “Kₐ”, as used herein, is intended to refer to the equilibrium association constant of a particular antibody-antigen interaction. This constant has units of 1/M.

The term “K_{d}”, as used herein, is intended to refer to the equilibrium dissociation constant of a particular antibody-antigen interaction. This constant has units of M.

The term “kₐ”, as used herein, is intended to refer to the kinetic association constant of a particular antibody-antigen interaction. This constant has units of 1/Ms.

The term “k_{d}”, as used herein, is intended to refer to the kinetic dissociation constant of a particular antibody-antigen interaction. This constant has units of 1/s.

“Particular antibody-antigen interactions” refers to the experimental conditions under which the equilibrium and kinetic constants are measured.

“Isotype” refers to the antibody class that is encoded by heavy chain constant region genes. Heavy chains are classified as gamma, mu, alpha, delta, or epsilon, and define the antibody's isotype as IgG, IgM, IgA, IgD and IgE, respectively. Additional structural variations characterize distinct subtypes of IgG (e.g., IgG₁, IgG₂, IgG₃ and IgG₄) and IgA (e.g., IgA₁ and IgA₂)

“Isotype switching” refers to the phenomenon by which the class, or isotype, of an antibody changes from one Ig class to one of the other Ig classes.

“Nonswitched isotype” refers to the isotypic class of heavy chain that is produced when no isotype switching has taken place; the C_{H} gene encoding the nonswitched isotype is typically the first C_{H} gene immediately downstream from the functionally rearranged VDJ gene. Isotype switching has been classified as classical or non-classical isotype switching. Classical isotype switching occurs by recombination events which involve at least one switch sequence region in the transgene. Non-classical isotype switching can occur by, for example, homologous recombination between human σ_{μ} and human τ_{μ} (δ-associated deletion). Alternative non-classical switching mechanisms, such as intertransgene and/or interchromosomal recombination, among others, can occur and effectuate isotype switching.

“Switch sequence” refers to those DNA sequences responsible for switch recombination. A “switch donor” sequence, typically a μ switch region, are 5′ (i.e., upstream) of the construct region to be deleted during the switch recombination. The “switch acceptor” region are between the construct region to be deleted and the replacement constant region (e.g., γ, ε, and alike). As there is no specific site where recombination always occurs, the final gene sequence is not typically predictable from the construct.

“Glycosylation pattern” is defined as the pattern of carbohydrate units that are covalently attached to a protein, more specifically to an immunoglobulin protein. A glycosylation pattern of a heterologous antibody can be characterized as being substantially similar to glycosylation patterns which occur naturally on antibodies produced by the species of the non-human transgenic animal, when one of ordinary skill in the art would recognize the glycosylation pattern of the heterologous antibody as being more similar to said pattern of glycosylation in the species of the non-human transgenic animal than to the species from which the C_{H} genes of the transgene were derived.

“Naturally-occurring” as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

“Immunoglobulin locus” refers to a genetic element or set of linked genetic elements that comprise information that can be used by a B cell or B cell precursor to express an immunoglobulin peptide. This peptide can be a heavy chain peptide, a light chain peptide, or the fusion of a heavy and a light chain peptide. In the case of an unrearranged locus, the genetic elements are assembled by a B cell precursor to form the gene encoding an immunoglobulin peptide. In the case of a rearranged locus, a gene encoding an immunoglobulin peptide is contained within the locus.

“Rearranged” refers to a configuration of a heavy chain or light chain immunoglobulin locus wherein a V segment is positioned immediately adjacent to a D-J or J segment in a conformation encoding essentially a complete V_{H} or V_{L} domain, respectively. A rearranged immunoglobulin gene locus can be identified by comparison to germline DNA; a rearranged locus has at least one recombined heptamer/nonamer homology element.

“Unrearranged” or “germline configuration” in reference to a V segment refers to the configuration wherein the V segment is not recombined so as to be immediately adjacent to a D or J segment.

“Nucleic acid” or “nucleic acid molecule” refer to a deoxyribonucleotide or ribonucleotide polymer in either single- or double-stranded form, and unless otherwise limited, can encompass known analogs of natural nucleotides that can function in a similar manner as naturally occurring nucleotides.

“Isolated nucleic acid” in reference to nucleic acids encoding antibodies or antibody portions (e.g., V_{H}, V_{L}, CDR3) that bind to the antigen, is intended to refer to a nucleic acid in which the nucleotide sequences encoding the antibody or antibody portion are free of other nucleotide sequences encoding antibodies or antibody portions that bind antigens other than, for example, Chlamydia protease, which other sequences can naturally flank the nucleic acid in human genomic DNA.

“Substantially identical,” in the context of two nucleic acids or polypeptides refers to two or more sequences or subsequences that have at least about 80%, about 90%, about 95% or higher nucleotide or amino acid residue identity, when compared and aligned for maximum correspondence, as measured using the following sequence comparison method and/or by visual inspection. Such “substantially identical” sequences are typically considered to be homologous. The “substantial identity” can exist over a region of sequence that is at least about 50 residues in length, over a region of at least about 100 residues, or over a region at least about 150 residues, or over the full length of the two sequences to be compared. As described below, any two antibody sequences can only be aligned in one way, by using the numbering scheme in Kabat. Therefore, for antibodies, percent identity has a unique and well-defined meaning.

Amino acids from the variable regions of the mature heavy and light chains of immunoglobulins are designated Hx and Lx respectively, where x is a number designating the position of an amino acid according to the scheme of Kabat, Sequences of Proteins of Immunological Interest (National Institutes of Health, Bethesda, Md., 1987 and 1991). Kabat lists many amino acid sequences for antibodies for each subgroup, and lists the most commonly occurring amino acid for each residue position in that subgroup to generate a consensus sequence. Kabat uses a method for assigning a residue number to each amino acid in a listed sequence, and this method for assigning residue numbers has become standard in the field. Kabat's scheme is extendible to other antibodies not included in his compendium by aligning the antibody in question with one of the consensus sequences in Kabat by reference to conserved amino acids. The use of the Kabat numbering system readily identifies amino acids at equivalent positions in different antibodies. For example, an amino acid at the L50 position of a human antibody occupies the equivalent position to an amino acid position L50 of a mouse antibody. Likewise, nucleic acids encoding antibody chains are aligned when the amino acid sequences encoded by the respective nucleic acids are aligned according to the Kabat numbering convention. An alternative structural definition has been proposed by Chothia, et al., J. Mol. Biol. 196:901-917, 1987; Chothia, et al., Nature 342:878-883, 1989; and Chothia, et al., J. Mol. Biol. 186:651-663, 1989, which are herein incorporated by reference for all purposes.

The nucleic acids of the invention be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art (See, e.g., Sambrook et al., Molecular Cloning, A Laboratory Manual, 2^{nd} ed., 1989; Tijssen (1993); and Ausubel (1994), incorporated by reference for all purposes). The nucleic acid sequences of the invention and other nucleic acids used to practice this invention, whether RNA, cDNA, genomic DNA, or hybrids thereof, can be isolated from a variety of sources, genetically engineered, amplified, and/or expressed recombinantly. Any recombinant expression system can be used, including, in addition to bacterial, e.g., yeast, insect or mammalian systems. Alternatively, these nucleic acids can be chemically synthesized in vitro. Techniques for the manipulation of nucleic acids, such as, e.g., subcloning into expression vectors, labeling probes, sequencing, and hybridization are well described in the scientific and patent literature, see, e.g., Sambrook, et al., 1989. Nucleic acids can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, such as fluid or gel precipitin reactions, immunodiffusion (single or double), immunoelectrophoresis, radioimmunoassay (RIAs), enzyme-linked immunosorbent assays (ELISAs), immuno-fluorescent assays, Southern analysis, Northern analysis, dot-blot analysis, gel electrophoresis (e.g., SDS-PAGE), RT-PCR, quantitative PCR, other nucleic acid or target or signal amplification methods, radiolabeling, scintillation counting, and affinity chromatography.

The nucleic acid compositions of the present invention, while often in a native sequence (except for modified restriction sites and the like), from either cDNA, genomic or mixtures can be mutated, thereof in accordance with standard techniques to provide gene sequences. For coding sequences, these mutations, can affect amino acid sequence as desired. In particular, DNA sequences substantially homologous to or derived from native V, D, J, constant, switches and other such sequences described herein are contemplated (where “derived” indicates that a sequence is identical or modified from another sequence).

“Recombinant host cell” or “host cell” refers to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

A “label” is a composition detectable by spectroscopic, photochemical, biochemical, immunochemical, or chemical means. For example, useful labels include ³²P, fluorescent dyes, electron-dense reagents, enzymes (e.g., as commonly used in an ELISA), biotin, digoxigenin, or haptens and proteins for which antisera or monoclonal antibodies are available (e.g., the polypeptides of the invention can be made detectable, e.g., by incorporating a radiolabel into the peptide, and used to detect antibodies specifically reactive with the peptide).

“Sorting” in the context of cells as used herein to refers to both physical sorting of the cells, as can be accomplished using, e.g., a fluorescence activated cell sorter, as well as to analysis of cells based on expression of cell surface markers, e.g., FACS analysis in the absence of sorting.

Components of an immune response can be detected in vitro by various methods that are well known to those of ordinary skill in the art. For example, (1) cytotoxic T lymphocytes can be incubated with radioactively labeled target cells and the lysis of these target cells detected by the release of radioactivity, (2) helper T lymphocytes can be incubated with antigens and antigen presenting cells and the synthesis and secretion of cytokines and proliferation as measured by assays described below and measured by standard methods (Windhagen A; et al., Immunity 2:373-380, 1995), (3) antigen presenting cells can be incubated with whole protein antigen and the presentation of that antigen on MHC detected by either T lymphocyte activation assays or biophysical methods (Harding et al., Proc. Natl. Acad. Sci. U.S.A., 86:4230-4, 1989), (4) mast cells can be incubated with reagents that cross-link their Fc-epsilon receptors and histamine release measured by enzyme immunoassay (Siraganian, et al., TIPS 4:432-437, 1983).

Similarly, products of an immune response in either a model organism (e.g., mouse) or a human patient can also be detected by various methods that are well known to those of ordinary skill in the art. For example, (1) the production of antibodies in response to vaccination can be readily detected by standard methods currently used in clinical laboratories, e.g., an ELISA; (2) the migration of immune cells to sites of inflammation can be detected by scratching the surface of skin and placing a sterile container to capture the migrating cells over scratch site (Peters et al., Blood 72:1310-5, 1988); (3) the proliferation of peripheral blood mononuclear cells in response to mitogens or mixed lymphocyte reaction can be measured using ³H-thymidine; (4) the phagocytic capacity of granulocytes, macrophages, and other phagocytes in PBMCs can be measured by placing PMBCs in wells together with labeled particles (Peters et al., 1988); and (5) the radioimmunoassay of immune system cells can be measured by labeling PBMCs with antibodies to CD molecules such as CD4 and CD8 and measuring the fraction of the PBMCs expressing these markers.

“Signal transduction pathway” or “signal transduction event” refers to at least one biochemical reaction, but more commonly a series of biochemical reactions, which result from interaction of a cell with a stimulatory compound or agent, e.g., NF-κB signal transduction. Thus, the interaction of a stimulatory compound with a cell generates a “signal” that is transmitted through the signal transduction pathway, ultimately resulting in a cellular response, e.g., an immune response described above.

A signal transduction pathway refers to the biochemical relationship between a variety of signal transduction molecules that play a role in the transmission of a signal from one portion of a cell to another portion of a cell. As used herein, the phrase “cell surface receptor” includes molecules and complexes of molecules capable of receiving a signal and the transmission of such a signal across the plasma membrane of a cell. An example is NF-κB signal transduction, and Chlamydia tail-specific protease cleavage of p65/RelA to inhibit NF-κB signal transduction.

A signal transduction pathway in a cell can be initiated by interaction of a cell with a stimulator that is inside or outside of the cell. If an exterior (i.e., outside of the cell) stimulator (e.g., an MHC-antigen complex on an antigen presenting cell with a polypeptide epitope of Chlamydia tail-specific protease) interacts with a cell surface receptor (e.g., a T cell receptor), a signal transduction pathway can transmit a signal across the cell's membrane, through the cytoplasm of the cell, and in some instances into the nucleus. If an interior (e.g., inside the cell) stimulator interacts with an intracellular signal transduction molecule, a signal transduction pathway can result in transmission of a signal through the cell's cytoplasm, and in some instances into the cell's nucleus.

Signal transduction can occur through, e.g., the phosphorylation of a molecule; non-covalent allosteric interactions; complexing of molecules; change of protein localization; the conformational change of a molecule; calcium release; inositol phosphate production; proteolytic cleavage; cyclic nucleotide production and diacylglyceride production. Typically, signal transduction occurs through phosphorylating a signal transduction molecule.

“Nonspecific T cell activation” refers to the stimulation of T cells independent of their antigenic specificity.

This invention relies on routine techniques in the field of recombinant genetics. Basic texts disclosing the general methods of use in this invention include Sambrook et al., Molecular Cloning, A Laboratory Manual, 2^{nd} ed., 1989; Kriegler, Gene Transfer and Expression: A Laboratory Manual, 1990; and Ausubel et al., eds., Current Protocols in Molecular Biology, 1994.

Chlamydia protease, e.g., tail specific protease CT441 or tail-specific protease Cpn0555, nucleic acids, polymorphic variants, orthologs, and alleles that are substantially identical to sequences provided herein can be isolated using nucleic acid probes and oligonucleotides of Chlamydia protease, e.g., tail specific protease CT441 or tail-specific protease Cpn0555, under stringent hybridization conditions, by screening libraries. Alternatively, expression libraries can be used to isolate Chlamydia protease, or protein encoding Chlamydia protease polymorphic variants, orthologs, and alleles by detecting expressed homologs immunologically with antisera or purified antibodies made against human Chlamydia protease, or portions thereof.

### Screening Methodologies

Methods for identifying compounds that inhibit Chlamydia infectious disease can identify compounds that interact with and inhibit a function of Chlamydia protease, e.g., tail specific protease CT441 or tail-specific protease Cpn0555. A function Chlamydia protease can include cleavage of p65 subunit of nuclear factor NF-κB. The present invention demonstrates that Chlamydia sp. has the ability to interfere with the NF-κB pathway of host inflammatory response. We found that Chlamydia infection did not promote IκBα degradation, a prerequisite for NF-κB nuclear translocation/activation, nor induce p65 nuclear redistribution. Instead, it caused p65 cleavage into an N-terminus-derived p40 fragment and a p22 of the C-terminus. The chlamydial protein that selectively cleaved p65 was identified as a tail-specific protease. Importantly, expression of either this protease or the p40 cleavage product could block NF-κB activation. Together, these data suggest that Chlamydia sp. has the ability to convert a regulatory molecule of host inflammatory response to a dominant negative inhibitor of the same pathway potentially to minimize inflammation.

A method for treating inflammation or inflammatory disease in a mammalian subject is provided which comprises administering an N-terminal p40 fragment of p65/RelA or an analog thereof, or a Chlamydia tail-specific protease or analog thereof, to the mammalian subject, wherein the p40 fragment or analog thereof is administered in an amount effective to reduce or eliminate the inflammation or inflammatory disease or to prevent its occurrence or recurrence.

In some embodiments, the test compounds bind to a Chlamydia protease polypeptide or nucleic acid, e.g., mRNA, and cause a decrease in levels of Chlamydia protease polypeptide.

These methods can be used to identify test compounds that inhibit Chlamydia protease function. In some embodiments, the methods include determining whether a compound can bind to Chlamydia protease and cause the inhibition of Chlamydia sp. replication in cells.

In some embodiments, the methods include determining whether a compound that is known to bind to Chlamydia protease also inhibits Chlamydia protease role in Chlamydia infectious disease or Chlamydia persistent infection.

In some embodiments, the methods include providing one or more samples that include both Chlamydia protease and one or more test compounds. An “active fragment” is a fragment that retains the ability to bind the other protein, e.g., an active fragment of Chlamydia protease retains the ability to cleavage of p65 subunit of nuclear factor NF-κB.

A number of suitable assay methods to detect binding of test compounds to Chlamydia protease, e.g., tail specific protease CT441 or tail-specific protease Cpn0555, are known in the art and described herein, and include, but are not limited to, surface plasmon resonance (SPR)/Biacore™, fluorogenic binding assays, fluid phase binding assays, affinity chromatography, size exclusion or gel filtration, ELISA, immunoprecipitation, competitive binding assays, gel shift assays, and mass spectrometry based methods.

In some embodiments, methods described herein include a first screen for compounds that bind to Chlamydia protease, e.g., tail specific protease CT441 or tail-specific protease Cpn0555. Compounds that are identified as binding to Chlamydia protease can then be used in a second screen to identify those compounds that inhibit a function of Chlamydia protease. Alternatively, the first screen can be omitted and the compounds can simply be screened for their ability to inhibit a function of Chlamydia protease, e.g., to inhibit Chlamydia infection or replication in cells.

Once a compound that inhibits an action of Chlamydia protease is identified, the compound can be considered a candidate compound for the treatment of Chlamydia infectious disease. The ability of such compounds to treat infectious disease can be evaluated in a population of viable cells or in an animal, e.g., an animal model. A number of methods are known in the art and described herein for measuring Chlamydia infection by binding, entry, or replication in cells.

Such compounds are useful, e.g., as candidate therapeutic compounds for the treatment of Chlamydia infectious disease. Thus, included herein are methods for screening for candidate therapeutic compounds for the treatment of infectious disease, as described herein. The methods include administering the compound to a model of the condition, e.g., contacting a cell (in vitro) model with the compound, or administering the compound to an animal model of the condition, e.g., an animal model of a condition associated with Chlamydia infectious disease. The model is then evaluated for an effect of the candidate compound on the rate of Chlamydia infection of a model in vitro cell line, and a candidate compound that decrease the rate of Chlamydia infection of a model in vitro cell line can be considered a candidate therapeutic compound for the treatment of the condition. Such effects can include clinically relevant effects such as decreased Chlamydia infection as measured by binding, entry, or replication in cells. Such effects can be determined on a macroscopic or microscopic scale. Methods are such as those described herein. Candidate therapeutic compounds identified by these methods can be further verified, e.g., by administration to human subjects in a clinical trial.

The test compounds utilized in the assays and methods described herein can be, for example, nucleic acids, small molecules, organic or inorganic compounds, antibodies or antigen-binding fragments thereof, polynucleotides, peptides, or polypeptides. For example, Chlamydia protease, e.g., tail specific protease CT441 or tail-specific protease Cpn0555, polypeptides or polynucleotides (e.g., Chlamydia protease polypeptide variants including truncation mutants, deletion mutants, and point mutants; nucleic acids including sense, antisense, aptamers, and small inhibitory RNAs (siRNAs) including short hairpin RNAs (shRNAs) and ribozymes) can be used as test compounds in the methods described herein. Alternatively, compounds or compositions that mimic the Chlamydia protease can be used to induce a cell mediated immune response in an individual having a chlamydial infectious disease. A test compound that has been screened by an in vitro method described herein and determined to have a desired activity, e.g., inhibition of Chlamydia binding, entry, or replication in cells, can be considered a candidate compound. A candidate compound that has been screened, e.g., in an in vitro or in vivo model, and determined to have a desirable effect on one or more symptoms of a disorder associated with Chlamydia infectious disease, can be considered a candidate therapeutic agent. Candidate therapeutic agents, once screened in a clinical setting, are therapeutic agents, and both types of agents can be optionally optimized (e.g., by derivatization), and formulated with pharmaceutically acceptable excipients or carriers to form pharmaceutical compositions.

Small chemical molecule test compounds can initially be members of an organic or inorganic chemical library. As used herein, “small molecules” refers to small organic or inorganic molecules of molecular weight below about 3,000 Daltons. The small molecules can be natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio., 1: 60, 1997. In addition, a number of small molecule libraries are commercially available.

The test compound can have a structure that is based on an active fragment of Chlamydia protease. For example, computer modeling methods known in the can be used to rationally design a molecule that has a structure similar to an active fragment of Chlamydia protease.

In some embodiments, the compounds are optimized to improve their therapeutic index, i.e., increase therapeutic efficacy and/or decrease unwanted side effects. Thus, in some embodiments, the methods described herein include optimizing the test or candidate compound. In some embodiments, the methods include formulating a therapeutic composition including a test or candidate compound (e.g., an optimized compound) and a pharmaceutically acceptable carrier. In some embodiments, the compounds are optimized by derivatization using methods known in the art.

In some embodiments, the test compound comprises a polynucleotide that encodes Chlamydia protease, e.g., tail specific protease CT441 or tail-specific protease Cpn0555, or an active fragment thereof. In some embodiments, the compound is a polynucleotide that encodes an active or inactive fragment of Chlamydia protease.

In some embodiments, the test compound comprises a polynucleotide that encodes a polypeptide that is at least about 85% identical to the amino acid sequence of Chlamydia protease, e.g., tail specific protease CT441 or tail-specific protease Cpn0555. In some embodiments, the polynucleotide encodes a polypeptide that is at least about 90%, 95%, 99%, or 100% identical to the full length sequence of a Chlamydia protease or an active fragment thereof. In some embodiments, the polynucleotide encodes an active peptide fragment thereof that retains the ability to inhibit Chlamydia infection by inhibition of binding, entry, or replication in cells. In some embodiments, the active fragment is at least about 10, 12, 14, 16, 18, 20, 22, 24, 26, 28, 30, 32, 34, 36, 38, 39, 40, 45, 50, 55, 60, 65, 70, 75, 80 or more amino acids long. The nucleic acid can include one or more noncoding regions of the coding strand of a nucleotide sequence encoding Chlamydia protease (e.g., the 5′ and 3′ untranslated regions). A number of methods are known in the art for obtaining suitable nucleic acids, see, e.g., Sambrook et al., Molecular Cloning: A Laboratory Manual (Cold Spring Harbor Laboratory, Cold Spring Harbor, N.Y.; 3rd ed. 2001).

In practicing the methods of the invention, a variety of apparatus and methodologies can be used to in conjunction with the polypeptides and nucleic acids of the invention, e.g., to screen polypeptides for Chlamydia protease activity, to screen compounds as potential modulators (e.g., inhibitors) of a Chlamydia protease activity, for antibodies that bind to a polypeptide of the invention, for nucleic acids that hybridize to a nucleic acid of the invention, to screen for cells expressing a polypeptide of the invention and the like.

In one aspect, the peptides and polypeptides of the invention can be bound to a solid support. Solid supports can include, e.g., membranes (e.g., nitrocellulose or nylon), a microtiter dish (e.g., PVC, polypropylene, or polystyrene), a test tube (glass or plastic), a dip stick (e.g., glass, PVC, polypropylene, polystyrene, latex and the like), a microfuge tube, or a glass, silica, plastic, metallic or polymer bead or other substrate such as paper. One solid support uses a metal (e.g., cobalt or nickel)-comprising column which binds with specificity to a histidine tag engineered onto a peptide.

Adhesion of peptides to a solid support can be direct (i.e., the protein contacts the solid support) or indirect (a particular compound or compounds are bound to the support and the target protein binds to this compound rather than the solid support). Peptides can be immobilized either covalently (e.g., utilizing single reactive thiol groups of cysteine residues (see, e.g., Colliuod, Bioconjugate Chem. 4: 528-536, 1993) or non-covalently but specifically (e.g., via immobilized antibodies (see, e.g., Schuhmann, Adv. Mater. 3: 388-391, 1991; Lu, Anal. Chem. 67: 83-87, 1995; the biotin/strepavidin system (see, e.g., Iwane, Biophys. Biochem. Res. Comm. 230: 76-80, 1997); metal chelating, e.g., Langmuir-Blodgett films (see, e.g., Ng, Langmuir 11: 4048-55, 1995); metal-chelating self-assembled monolayers (see, e.g., Sigal, Anal. Chem. 68: 490-497, 1996) for binding of polyhistidine fusions.

Indirect binding can be achieved using a variety of linkers which are commercially available. The reactive ends can be any of a variety of functionalities including, but not limited to: amino reacting ends such as N-hydroxysuccinimide (NHS) active esters, imidoesters, aldehydes, epoxides, sulfonyl halides, isocyanate, isothiocyanate, and nitroaryl halides; and thiol reacting ends such as pyridyl disulfides, maleimides, thiophthalimides, and active halogens. The heterobifunctional crosslinking reagents have two different reactive ends, e.g., an amino-reactive end and a thiol-reactive end, while homobifunctional reagents have two similar reactive ends, e.g., bismaleimidohexane (BMH) which permits the cross-linking of sulfhydryl-containing compounds. The spacer can be of varying length and be aliphatic or aromatic. Examples of commercially available homobifunctional cross-linking reagents include, but are not limited to, the imidoesters such as dimethyl adipimidate dihydrochloride (DMA); dimethyl pimelimidate dihydrochloride (DMP); and dimethyl suberimidate dihydrochloride (DMS). Heterobifunctional reagents include commercially available active halogen-NHS active esters coupling agents such as N-succinimidyl bromoacetate and N-succinimidyl (4-iodoacetyl)aminobenzoate (SLAB) and the sulfosuccinimidyl derivatives such as sulfosuccinimidyl(4-iodoacetyl)aminobenzoate (sulfo-SIAB) (Pierce). Another group of coupling agents is the heterobifunctional and thiol cleavable agents such as N-succinimidyl 3-(2-pyridyldithio)propiona-te (SPDP) (Pierce Chemicals, Rockford, Ill.).

Antibodies can be used for binding polypeptides and peptides of the invention to a solid support. This can be done directly by binding peptide-specific antibodies to the column or it can be done by creating fusion protein chimeras comprising motif-containing peptides linked to, e.g., a known epitope (e.g., a tag (e.g., FLAG, myc) or an appropriate immunoglobulin constant domain sequence (an “immunoadhesin,” see, e.g., Capon, Nature 377: 525-531, 1989.

### Screening by Microarrays or “Biochips”

The invention provides methods for identifying/screening for modulators (e.g., inhibitors) of Chlamydia protease, e.g., tail specific protease CT441 or tail-specific protease Cpn0555activity, using arrays. Potential inhibitors, including small molecules, nucleic acids, polypeptides (including antibodies) can be immobilized to arrays. Nucleic acids or polypeptides of the invention can be immobilized to or applied to an array. Arrays can be used to screen for or monitor libraries of compositions (e.g., small molecules, antibodies, or nucleic acids) for their ability to bind to or modulate the activity of a nucleic acid or a polypeptide of the invention, e.g., Chlamydia protease activity. For example, in one aspect of the invention, a monitored parameter is transcript expression of a gene comprising a nucleic acid of the invention. One or more, or, all the transcripts of a cell can be measured by hybridization of a sample comprising transcripts of the cell, or, nucleic acids representative of or complementary to transcripts of a cell, by hybridization to immobilized nucleic acids on an array, or “biochip.” By using an “array” of nucleic acids on a microchip, some or all of the transcripts of a cell can be simultaneously quantified. Alternatively, arrays comprising genomic nucleic acid can also be used to determine the genotype of a newly engineered strain made by the methods of the invention. Polypeptide arrays can be used to simultaneously quantify a plurality of proteins. Small molecule arrays can be used to simultaneously analyze a plurality of Chlamydia protease modulating or binding activities.

The present invention can be practiced with any known “array,” also referred to as a “microarray” or “nucleic acid array” or “polypeptide array” or “antibody array” or “biochip,” or variation thereof. Arrays are generically a plurality of “spots” or “target elements,” each target element comprising a defined amount of one or more biological molecules, e.g., oligonucleotides, immobilized onto a defined area of a substrate surface for specific binding to a sample molecule, e.g., mRNA transcripts. In practicing the methods of the invention, any known array and/or method of making and using arrays can be incorporated in whole or in part, or variations thereof, as described, for example, in U.S. Pat. Nos. 6,277,628; 6,277,489; 6,261,776; 6,258,606; 6,054,270; 6,048,695; 6,045,996; 6,022,963; 6,013,440; 5,965,452; 5,959,098; 5,856,174; 5,830,645; 5,770,456; 5,632,957; 5,556,752; 5,143,854; 5,807,522; 5,800,992; 5,744,305; 5,700,637; 5,556,752; 5,434,049; see also, e.g., WO 99/51773; WO 99/09217; WO 97/46313; WO 96/17958; see also, e.g., Johnston, Curr. Biol. 8: R171-R174, 1998; Schummer, Biotechniques 23: 1087-1092, 1997; Kern, Biotechniques 23: 120-124, 1997; Solinas-Toldo, Genes, Chromosomes & Cancer 20: 399-407, 1997; Bowtell, Nature Genetics Supp. 21: 25-32, 1999. See also published U.S. patent applications Nos. 20010018642; 20010019827; 20010016322; 20010014449; 20010014448; 20010012537; 20010008765.

The terms “array” or “microarray” or “biochip” or “chip” as used herein is a plurality of target elements, each target element comprising a defined amount of one or more polypeptides (including antibodies) or nucleic acids immobilized onto a defined area of a substrate surface.

### Combinatorial Chemical Libraries

The invention provides methods for identifying/screening for modulators (e.g., inhibitors) of a Chlamydia protease activity, e.g., tail specific protease CT441 activity or tail-specific protease Cpn0555 activity. In practicing the screening methods of the invention, a test compound is provided. It can be contacted with a polypeptide of the invention in vitro or administered to a cell of the invention or an animal of the invention in vivo. Compounds are also screened using the compositions, cells, non-human animals and methods of the invention for their ability to treat or ameliorate a Chlamydia infectious disease in an animal. Combinatorial chemical libraries are one means to assist in the generation of new chemical compound leads for, e.g., compounds that inhibit an Chlamydia protease activity of the invention, or a compound that can be used to treat or ameliorate a Chlamydia infectious disease.

A combinatorial chemical library is a collection of diverse chemical compounds generated by either chemical synthesis or biological synthesis by combining a number of chemical “building blocks” such as reagents. For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, the systematic, combinatorial mixing of 100 interchangeable chemical building blocks results in the theoretical synthesis of 100 million tetrameric compounds or 10 billion pentameric compounds (see, e.g., Gallop et al. (1994) 37(9): 1233-1250). Preparation and screening of combinatorial chemical libraries are well known to those of skill in the art, see, e.g., U.S. Pat. Nos. 6,004,617; 5,985,356. Such combinatorial chemical libraries include, but are not limited to, peptide libraries (see, e.g., U.S. Pat. No. 5,010,175; Furka, Int. J. Pept. Prot. Res. 37: 487-493, 1991, Houghton et al. Nature, 354: 84-88, 1991). Other chemistries for generating chemical diversity libraries include, but are not limited to: peptoids (see, e.g., WO 91/19735), encoded peptides (see, e.g., WO 93/20242), random bio-oligomers (see, e.g., WO 92/00091), benzodiazepines (see, e.g., U.S. Pat. No. 5,288,514), diversomers such as hydantoins, benzodiazepines and dipeptides (see, e.g., Hobbs, Proc. Nat. Acad. Sci. USA 90: 6909-6913, 1993), vinylogous polypeptides (see, e.g., Hagihara, J. Amer. Chem. Soc. 114: 6568, 1992), non-peptidal peptidomimetics with a Beta-D-Glucose scaffolding (see, e.g., Hirschmann, J. Amer. Chem. Soc. 114: 9217-9218, 1992), analogous organic syntheses of small compound libraries (see, e.g., Chen, J. Amer. Chem. Soc. 116: 2661, 1994), oligocarbamates (see, e.g., Cho, Science 261: 1303, 1993), and/or peptidyl phosphonates (see, e.g., Campbell, J. Org. Chem. 59: 658, 1994). See also Gordon, J. Med. Chem. 37: 1385, 1994; for nucleic acid libraries, peptide nucleic acid libraries, see, e.g., U.S. Pat. No. 5,539,083; for antibody libraries, see, e.g., Vaughn, Nature Biotechnology 14: 309-314, 1996; for carbohydrate libraries, see, e.g., Liang et al. Science 274: 1520-1522, 1996, U.S. Pat. No. 5,593,853; for small organic molecule libraries, see, e.g., for isoprenoids U.S. Pat. No. 5,569,588; for thiazolidinones and metathiazanones, U.S. Pat. No. 5,549,974; for pyrrolidines, U.S. Pat. Nos. 5,525,735 and 5,519,134; for morpholino compounds, U.S. Pat. No. 5,506,337; for benzodiazepines U.S. Pat. No. 5,288,514.

Devices for the preparation of combinatorial libraries are commercially available (see, e.g., U.S. Pat. Nos. 6,045,755; 5,792,431; 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). A number of robotic systems have also been developed for solution phase chemistries. These systems include automated workstations, e.g., like the automated synthesis apparatus developed by Takeda Chemical Industries, LTD. (Osaka, Japan) and many robotic systems utilizing robotic arms (Zymate II, Zymark Corporation, Hopkinton, Mass; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

### Antibodies and Antibody-Based Screening Methods

The invention provides isolated or recombinant antibodies that specifically bind to a polypeptide or nucleic acid of the invention Chlamydia protease polypeptides e.g., tail specific protease CT441 or tail-specific protease Cpn0555 polypeptides, or an N-terminal p40 fragment of p65/RelA or an analog or peptidomimetic thereof. These antibodies can be used as therapeutic antibodies to treat disease or to isolate, identify or quantify a polypeptide of the invention or related polypeptides. These antibodies can be used to isolate other polypeptides within the scope the invention that act as inhibitors of Chlamydia infection in pathways related to entry or replication of Chlamydia in the cells.

The term “antibody” includes a peptide or polypeptide derived from, modeled after or substantially encoded by an immunoglobulin gene or immunoglobulin genes, or fragments thereof, capable of specifically binding an antigen or epitope, see, e.g. Fundamental Immunology, Third Edition, W. E. Paul, ed., Raven Press, N.Y., 1993; Wilson, J. Immunol. Methods 175: 267-273, 1994; Yarmush, J. Biochem. Biophys. Methods 25: 85-97, 1992. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the V_{L}, V_{H}, C_{L} and C_{H}1 domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., Nature 341: 544-546, 1989), which consists of a V_{H} domain; and (vi) an isolated complementarity determining region (CDR). Single chain antibodies are also included by reference in the term “antibody.”

The antibodies can be used in immunoprecipitation, staining (e.g., FACS), immunoaffinity columns, and the like. If desired, nucleic acid sequences encoding for specific antigens can be generated by immunization followed by isolation of polypeptide or nucleic acid, amplification or cloning and immobilization of polypeptide onto an array of the invention. Alternatively, the methods of the invention can be used to modify the structure of an antibody produced by a cell to be modified, e.g., an antibody's affinity can be increased or decreased. Furthermore, the ability to make or modify antibodies can be a phenotype engineered into a cell by the methods of the invention.

Methods of immunization, producing and isolating antibodies (polyclonal and monoclonal) are known to those of skill in the art and described in the scientific and patent literature, see, e.g., Coligan, CURRENT PROTOCOLS IN IMMUNOLOGY, Wiley/Greene, NY (1991); Stites (eds.) BASIC AND CLINICAL IMMUNOLOGY (7^{th} ed.) Lange Medical Publications, Los Altos, Calif. (“Stites”); Goding, MONOCLONAL ANTIBODIES: PRINCIPLES AND PRACTICE (2d ed.) Academic Press, New York, N.Y., 1986; Kohler, Nature 256:495, 1975; Harlow, ANTIBODIES, A LABORATORY MANUAL, Cold Spring Harbor Publications, New York, 1988. Antibodies also can be generated in vitro, e.g., using recombinant antibody binding site expressing phage display libraries, in addition to the traditional in vivo methods using animals. See, e.g., Hoogenboom, Trends Biotechnol. 15: 62-70, 1997; Katz, Annu. Rev. Biophys. Biomol. Struct. 26: 27-45, 1997.

Polypeptides or peptides can be used to generate antibodies which bind specifically to the polypeptides of the invention. The resulting antibodies can be used in immunoaffinity chromatography procedures to isolate or purify the polypeptide or to determine whether the polypeptide is present in a biological sample. In such procedures, a protein preparation, such as an extract, or a biological sample is contacted with an antibody capable of specifically binding to one of the polypeptides of the invention.

In immunoaffinity procedures, the antibody is attached to a solid support, such as a bead or other column matrix. The protein preparation is placed in contact with the antibody under conditions in which the antibody specifically binds to one of the polypeptides of the invention. After a wash to remove non-specifically bound proteins, the specifically bound polypeptides are eluted.

The ability of proteins in a biological sample to bind to the antibody can be determined using any of a variety of procedures familiar to those skilled in the art. For example, binding can be determined by labeling the antibody with a detectable label such as a fluorescent agent, an enzymatic label, or a radioisotope. Alternatively, binding of the antibody to the sample can be detected using a secondary antibody having such a detectable label thereon. Particular assays include ELISA assays, sandwich assays, radioimmunoassay, and Western Blots.

Polyclonal antibodies generated against the polypeptides of the invention can be obtained by direct injection of the polypeptides into an animal or by administering the polypeptides to a non-human animal. The antibody so obtained will then bind the polypeptide itself. In this manner, even a sequence encoding only a fragment of the polypeptide can be used to generate antibodies which can bind to the whole native polypeptide. Such antibodies can then be used to isolate the polypeptide from cells expressing that polypeptide.

For preparation of monoclonal antibodies, any technique which provides antibodies produced by continuous cell line cultures can be used. Examples include the hybridoma technique, the trioma technique, the human B-cell hybridoma technique, and the EBV-hybridoma technique (see, e.g., Cole (1985) in Monoclonal Antibodies and Cancer Therapy, Alan R. Liss, Inc., pp. 77-96).

Techniques described for the production of single chain antibodies (see, e.g., U.S. Pat. No. 4,946,778) can be adapted to produce single chain antibodies to the polypeptides of the invention. Alternatively, transgenic mice can be used to express humanized antibodies to these polypeptides or fragments thereof.

Antibodies generated against the polypeptides of the invention can be used in screening for similar polypeptides from other organisms and samples. In such techniques, polypeptides from the organism are contacted with the antibody and those polypeptides which specifically bind the antibody are detected. Any of the procedures described above can be used to detect antibody binding.

### Peptides and Polypeptides

The invention provides isolated or recombinant polypeptides comprising an amino acid sequence having at least 95%, 96%, 97%, 98%, 99% or more sequence identity to SEQ ID NO:2 or SEQ ID NO:5 over a region of at least about 100, 150, 200, 250, 300, 350, 400, 450, 500, 550, 600, 650, 700, 750, 800, 850, 900, 950, 1000, 1100 or more residues, or, the full length of the polypeptide, or, a polypeptide encoded by a nucleic acid of the invention. The invention provides methods for inhibiting the activity of Chlamydia protease polypeptide, e.g., tail specific protease CT441 or tail-specific protease Cpn0555 polypeptides, that is, a polypeptide of the invention. The invention also provides methods for screening for compositions that inhibit the activity of, or bind to (e.g., bind to the active site), of Chlamydia protease polypeptides, e.g., a polypeptide of the invention.

In one aspect, the invention provides Chlamydia protease polypeptides (and the nucleic acids encoding them) where one, some or all of the Chlamydia protease polypeptides replacement with substituted amino acids. In one aspect, the invention provides methods to disrupt the interaction of Chlamydia protease polypeptides with other proteins, in pathways related to entry or replication of infectious viruses in the cells.

The peptides and polypeptides of the invention can be expressed recombinantly in vivo after administration of nucleic acids, as described above, or, they can be administered directly, e.g., as a pharmaceutical composition. They can be expressed in vitro or in vivo to screen for modulators of a Chlamydia protease activity and for agents that can treat or ameliorate a Chlamydia infectious disease.

Polypeptides and peptides of the invention can be isolated from natural sources, be synthetic, or be recombinantly generated polypeptides. Peptides and proteins can be recombinantly expressed in vitro or in vivo. The peptides and polypeptides of the invention can be made and isolated using any method known in the art. Polypeptide and peptides of the invention can also be synthesized, whole or in part, using chemical methods well known in the art. See e.g., Caruthers, Nucleic Acids Res. Symp. Ser. 215-223, 1980; Horn, Nucleic Acids Res. Symp. Ser. 225-232, 1980; Banga, A. K., Therapeutic Peptides and Proteins, Formulation, Processing and Delivery Systems Technomic Publishing Co., Lancaster, Pa., 1995. For example, peptide synthesis can be performed using various solid-phase techniques (see e.g., Roberge, Science 269: 202, 1995; Merrifield, Methods Enzymol. 289: 3-13, 1997) and automated synthesis can be achieved, e.g., using the ABI 431A Peptide Synthesizer (Perkin Elmer) in accordance with the instructions provided by the manufacturer.

The peptides and polypeptides of the invention, as defined above, include all “mimetic” and “peptidomimetic” forms. The terms “mimetic” and “peptidomimetic” refer to a synthetic chemical compound which has substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. As with polypeptides of the invention which are conservative variants, routine experimentation will determine whether a mimetic is within the scope of the invention, i.e., that its structure and/or function is not substantially altered. Thus, a mimetic composition is within the scope of the invention if, when administered to or expressed in a cell, it has a Chlamydia protease activity.

In one aspect, the polypeptide or peptidomimetic composition can be a dominant-negative mutant within the scope of the invention if it can block NF-κB activation and reduce or eliminate inflammation or inflammatory disease or prevent its occurrence or recurrence in a mammalian subject. For example, an N-terminal p40 fragment of p65/RelA or an analog or peptidomimetic thereof can block NF-κB activation. A dominant negative peptide can inhibit an activity of a Chlamydia protease polypeptide, e.g., tail specific protease CT441 or tail-specific protease Cpn0555 polypeptides of the invention, e.g., be a dominant-negative mutant or bind to an antibody of the invention. The dominant negative mutant can be a peptide or peptide mimetic that can inhibit an activity of a Chlamydia protease, or a nucleic acid composition, in the form of a DNA vector or gene therapy vector, that expresses a dominant-negative polypeptide that can can block NF-κB activation and prevent inflammation or inflammatory disease or inhibit an activity of a Chlamydia protease. The dominant negative mutant can bind to a ligand of the Chlamydia protease or a target, affecting ligand target interaction. The dominant negative molecule can act, for example, by blocking protein-protein interactions, or by blocking phosphorylation of the Chlamydia protease. An example of a dominant negative peptide is a peptide with a mutation an N-terminal p40 fragment of p65/RelA or an analog or peptidomimetic thereof, as described herein, that reduces or prevents inflammation or inflammatory disease.

Polypeptide mimetic compositions can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, NY).

A polypeptide can also be characterized as a mimetic by containing all or some non-natural residues in place of naturally occurring amino acid residues. Non-natural residues are well described in the scientific and patent literature; a few exemplary non-natural compositions useful as mimetics of natural amino acid residues and guidelines are described below. Mimetics of aromatic amino acids can be generated by replacing by, e.g., D- or L-naphylalanine; D- or L-phenylglycine; D- or L-2 thieneylalanine; D- or L-1, -2,3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; K- or L-p-methoxy-biphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3(2-morpholin-yl-(4-ethyl) carbodiimide or 1-ethyl-3(4-azonia-4,4-dimetholpentyl) carbodiimide. Aspartyl or glutamyl can also be converted to asparaginyl and glutaminyl residues by reaction with ammonium ions.

Mimetics of basic amino acids can be generated by substitution with, e.g., (in addition to lysine and arginine) the amino acids ornithine, citrulline, or (guanidino)-acetic acid, or (guanidino)alkyl-acetic acid, where alkyl is defined above. Nitrile derivative (e.g., containing the CN-moiety in place of COOH) can be substituted for aspargine or glutamine. Asparaginyl and glutaminyl residues can be deaminated to the corresponding aspartyl or glutamyl residues.

Arginine residue mimetics can be generated by reacting arginyl with, e.g., one or more conventional reagents, including, e.g., phenylglyoxal, 2,3-butanedione, 1,2-cyclohexanedione, or ninhydrin, preferably under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitrobenzenesulfonic acid, O-methylisourea, 2,4, pentanedione, and transamidase-catalyzed reactions with glyoxylate. Mimetics of methionine can be generated by reaction with, e.g., methionine sulfoxide. Mimetics of □adioim include, e.g., pipecolic acid, thiazolidine carboxylic acid, 3- or 4-hydroxy guanidino, dehydroproline, 3- or 4-methylproline, or 3,3,-dimethylproline. Histidine residue mimetics can be generated by reacting histidyl with, e.g., diethylprocarbonate or para-bromophenacyl bromide. Other mimetics include, e.g., those generated by hydroxylation of guanidino and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

A component of a polypeptide of the invention can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, referred to as the D-amino acid, but which can additionally be referred to as the R- or S-form

The invention also provides polypeptides that are “substantially identical” to an exemplary polypeptide of the invention. A “substantially identical” amino acid sequence is a sequence that differs from a reference sequence by one or more conservative or non-conservative amino acid substitutions, deletions, or insertions, particularly when such a substitution occurs at a site that is not the active site of the molecule, and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for asparagine). One or more amino acids can be deleted, for example, from a Chlamydia protease polypeptide of the invention, resulting in modification of the structure of the polypeptide, without significantly altering its biological activity. For example, amino- or carboxyl-terminal, or internal, amino acids which are not required for a Chlamydia protease activity or interaction can be removed.

The skilled artisan will recognize that individual synthetic residues and polypeptides incorporating these mimetics can be synthesized using a variety of procedures and methodologies, which are well described in the scientific and patent literature, e.g., Organic Syntheses Collective Volumes, Gilman, et al. (Eds) John Wiley & Sons, Inc., NY. Peptides and peptide mimetics of the invention can also be synthesized using combinatorial methodologies. Various techniques for generation of peptide and peptidomimetic libraries are well known, and include, e.g., multipin, tea bag, and split-couple-mix techniques; see, e.g., al-Obeidi, Mol. Biotechnol. 9: 205-223, 1998; Hruby, Curr. Opin. Chem. Biol. 1: 114-119, 1997; Ostergaard, Mol. Divers. 3: 17-27, 1997; Ostresh, Methods Enzymol. 267: 220-234, 1996. Modified peptides of the invention can be further produced by chemical modification methods, see, e.g., Belousov, Nucleic Acids Res. 25: 3440-3444, 1997; Frenkel, Free Radic. Biol. Med. 19: 373-380, 1995; Blommers, Biochemistry 33: 7886-7896, 1994.

Peptides and polypeptides of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Amgen Inc., Seattle Wash.). The inclusion of a cleavable linker sequences such as Factor Xa or enterokinase (Invitrogen, San Diego Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams, Biochemistry 34: 1787-1797, 1995; Dobeli, Protein Expr. Purif. 12: 404-14, 1998). The histidine residues facilitate detection and purification while the enterokinase cleavage site provides a means for purifying the epitope from the remainder of the fusion protein. Technology pertaining to vectors encoding fusion proteins and application of fusion proteins are well described in the scientific and patent literature, see e.g., Kroll, DNA Cell. Biol., 12: 441-53, 1993.

“Polypeptide” and “protein” as used herein, refer to amino acids joined to each other by peptide bonds or modified peptide bonds, i.e., peptide isosteres, and can contain modified amino acids other than the 20 gene-encoded amino acids. The term “polypeptide” also includes peptides and polypeptide fragments, motifs and the like. The term also includes glycosylated polypeptides. The peptides and polypeptides of the invention also include all “mimetic” and “peptidomimetic” forms, as described in further detail, below.

“Isolated” means that the material is removed from its original environment (e.g., the natural environment if it is naturally occurring). For example, a naturally occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment. As used herein, an isolated material or composition can also be a “purified” composition, i.e., it does not require absolute purity; rather, it is intended as a relative definition. Individual nucleic acids obtained from a library can be conventionally purified to electrophoretic homogeneity. In alternative aspects, the invention provides nucleic acids which have been purified from genomic DNA or from other sequences in a library or other environment by at least one, two, three, four, five or more orders of magnitude.

### RNA and DNA Interference Methods

A. Short Interfering RNAs (RNAi)

RNA interference (RNAi) is a mechanism of post-transcriptional gene silencing mediated by double-stranded RNA (dsRNA), which is distinct from antisense and ribozyme-based approaches (see Jain, Pharmacogenomics 5: 239-42, 2004 for a review of RNAi and siRNA). RNA interference is useful in a method for treating a Chlamydia infectious disease in a mammal by administering to the mammal a nucleic acid molecule (e.g., dsRNA) that hybridizes under stringent conditions to a Chlamydia protease target gene, and attenuates expression of said target gene. dsRNA molecules are believed to direct sequence-specific degradation of mRNA in cells of various types after first undergoing processing by an RNase III-like enzyme called DICER (Bernstein et al., Nature 409: 363, 2001) into smaller dsRNA molecules comprised of two 21 nt strands, each of which has a 5′ phosphate group and a 3′ hydroxyl, and includes a 19 nt region precisely complementary with the other strand, so that there is a 19 nt duplex region flanked by 2 nt-3′ overhangs. RNAi is thus mediated by short interfering RNAs (siRNA), which typically comprise a double-stranded region approximately 19 nucleotides in length with 1-2 nucleotide 3′ overhangs on each strand, resulting in a total length of between approximately 21 and 23 nucleotides. In mammalian cells, dsRNA longer than approximately 30 nucleotides typically induces nonspecific mRNA degradation via the interferon response. However, the presence of siRNA in mammalian cells, rather than inducing the interferon response, results in sequence-specific gene silencing.

In general, a short, interfering RNA (siRNA) comprises an RNA duplex that is preferably approximately 19 basepairs long and optionally further comprises one or two single-stranded overhangs or loops. An siRNA may comprise two RNA strands hybridized together, or may alternatively comprise a single RNA strand that includes a self-hybridizing portion. siRNAs may include one or more free strand ends, which may include phosphate and/or hydroxyl groups. siRNAs typically include a portion that hybridizes under stringent conditions with a target transcript. One strand of the siRNA (or, the self-hybridizing portion of the siRNA) is typically precisely complementary with a region of the target transcript, meaning that the siRNA hybridizes to the target transcript without a single mismatch. In certain embodiments of the invention in which perfect complementarity is not achieved, it is generally preferred that any mismatches be located at or near the siRNA termini.

siRNAs have been shown to downregulate gene expression when transferred into mammalian cells by such methods as transfection, electroporation, or microinjection, or when expressed in cells via any of a variety of plasmid-based approaches. RNA interference using siRNA is reviewed in, e.g., Tuschl, Nat. Biotechnol. 20: 446-448, 2002; See also Yu, J., et al., Proc. Natl. Acad. Sci., 99: 6047-6052, 2002; Sui, et al., Proc. Natl. Acad. Sci. USA. 99: 5515-5520, 2002; Paddison, et al., Genes and Dev. 16: 948-958, 2002; Brummelkamp, et al., Science 296: 550-553, 2002; Miyagashi, et al., Nat. Biotech. 20: 497-500, 2002; Paul, et al., Nat. Biotech. 20: 505-508, 2002. As described in these and other references, the siRNA may consist of two individual nucleic acid strands or of a single strand with a self-complementary region capable of forming a hairpin (stem-loop) structure. A number of variations in structure, length, number of mismatches, size of loop, identity of nucleotides in overhangs, etc., are consistent with effective siRNA-triggered gene silencing. While not wishing to be bound by any theory, it is thought that intracellular processing (e.g., by DICER) of a variety of different precursors results in production of siRNA capable of effectively mediating gene silencing. Generally it is preferred to target exons rather than introns, and it may also be preferable to select sequences complementary to regions within the 3′ portion of the target transcript. Generally it is preferred to select sequences that contain approximately equimolar ratio of the different nucleotides and to avoid stretches in which a single residue is repeated multiple times.

siRNAs may thus comprise RNA molecules having a double-stranded region approximately 19 nucleotides in length with 1-2 nucleotide 3′ overhangs on each strand, resulting in a total length of between approximately 21 and 23 nucleotides. As used herein, siRNAs also include various RNA structures that may be processed in vivo to generate such molecules. Such structures include RNA strands containing two complementary elements that hybridize to one another to form a stem, a loop, and optionally an overhang, preferably a 3′ overhang. Preferably, the stem is approximately 19 bp long, the loop is about 1-20, more preferably about 4-10, and most preferably about 6-8 nt long and/or the overhang is about 1-20, and more preferably about 2-15 nt long. In certain embodiments of the invention the stem is minimally 19 nucleotides in length and may be up to approximately 29 nucleotides in length. Loops of 4 nucleotides or greater are less likely subject to steric constraints than are shorter loops and therefore may be preferred. The overhang may include a 5′ phosphate and a 3′ hydroxyl. The overhang may but need not comprise a plurality of U residues, e.g., between 1 and 5 U residues. Classical siRNAs as described above trigger degradation of mRNAs to which they are targeted, thereby also reducing the rate of protein synthesis. In addition to siRNAs that act via the classical pathway, certain siRNAs that bind to the 3′ UTR of a template transcript may inhibit expression of a protein encoded by the template transcript by a mechanism related to but distinct from classic RNA interference, e.g., by reducing translation of the transcript rather than decreasing its stability. Such RNAs are referred to as microRNAs (mRNAs) and are typically between approximately 20 and 26 nucleotides in length, e.g., 22 nt in length. It is believed that they are derived from larger precursors known as small temporal RNAs (stRNAs) or mRNA precursors, which are typically approximately 70 nt long with an approximately 4-15 nt loop. (See Grishok, et al., Cell 106: 23-24, 2001; Hutvagner, et al., Science 293: 834-838, 2001; Ketting, et al., Genes Dev., 15: 2654-2659, 2001). Endogenous RNAs of this type have been identified in a number of organisms including mammals, suggesting that this mechanism of post-transcriptional gene silencing may be widespread (Lagos-Quintana, et al., Science 294: 853-858, 2001; Pasquinelli, Trends in Genetics 18: 171-173, 2002, and references in the foregoing two articles). MicroRNAs have been shown to block translation of target transcripts containing target sites in mammalian cells (Zeng, et al., Molecular Cell 9: 1-20, 2002).

siRNAs such as naturally occurring or artificial (i.e., designed by humans) mRNAs that bind within the 3′ UTR (or elsewhere in a target transcript) and inhibit translation may tolerate a larger number of mismatches in the siRNA/template duplex, and particularly may tolerate mismatches within the central region of the duplex. In fact, there is evidence that some mismatches may be desirable or required as naturally occurring stRNAs frequently exhibit such mismatches as do mRNAs that have been shown to inhibit translation in vitro. For example, when hybridized with the target transcript such siRNAs frequently include two stretches of perfect complementarity separated by a region of mismatch. A variety of structures are possible. For example, the mRNA may include multiple areas of nonidentity (mismatch). The areas of nonidentity (mismatch) need not be symmetrical in the sense that both the target and the mRNA include nonpaired nucleotides. Typically the stretches of perfect complementarity are at least 5 nucleotides in length, e.g., 6, 7, or more nucleotides in length, while the regions of mismatch may be, for example, 1, 2, 3, or 4 nucleotides in length.

Hairpin structures designed to mimic siRNAs and mRNA precursors are processed intracellularly into molecules capable of reducing or inhibiting expression of target transcripts (McManus, et al., RNA 8: 842-850, 2002). These hairpin structures, which are based on classical siRNAs consisting of two RNA strands forming a 19 bp duplex structure are classified as class I or class II hairpins. Class I hairpins incorporate a loop at the 5′ or 3′ end of the antisense siRNA strand (i.e., the strand complementary to the target transcript whose inhibition is desired) but are otherwise identical to classical siRNAs. Class II hairpins resemble mRNA precursors in that they include a 19 nt duplex region and a loop at either the 3′ or 5′ end of the antisense strand of the duplex in addition to one or more nucleotide mismatches in the stem. These molecules are processed intracellularly into small RNA duplex structures capable of mediating silencing. They appear to exert their effects through degradation of the target mRNA rather than through translational repression as is thought to be the case for naturally occurring mRNAs and stRNAs.

Thus it is evident that a diverse set of RNA molecules containing duplex structures is able to mediate silencing through various mechanisms. For the purposes of the present invention, any such RNA, one portion of which binds to a target transcript and reduces its expression, whether by triggering degradation, by inhibiting translation, or by other means, is considered to be an siRNA, and any structure that generates such an siRNA (i.e., serves as a precursor to the RNA) is useful in the practice of the present invention.

In the context of the present invention, siRNAs are useful both for therapeutic purposes, e.g., to modulate the expression of Chlamydia protease in a subject at risk of or suffering from a Chlamydia infectious disease and for various of the inventive methods for the identification of compounds for treatment of a Chlamydia infectious disease that modulate the activity or level of the molecules described herein. In a preferred embodiment, the therapeutic treatment of Chlamydia infectious disease, or persistent Chlamydia infectious disease, with an antibody, antisense vector, or double stranded RNA vector.

The invention therefore provides a method of inhibiting expression of a gene encoding a Chlamydia protease comprising the step of (i) providing a biological system in which expression of a gene encoding Chlamydia protease is to be inhibited; and (ii) contacting the system with an siRNA targeted to a transcript encoding the Chlamydia protease. According to certain embodiments of the invention the Chlamydia protease is encoded by a gene within Chlamydia bacterial genome. In other embodiments, Chlamydia proteases are inhibited. The Chlamydia proteases include, but are not limited to, tail specific protease CT441 or tail-specific protease Cpn0555. According to certain embodiments of the invention the biological system comprises a cell, and the contacting step comprises expressing the siRNA in the cell. According to certain embodiments of the invention the biological system comprises a subject, e.g., a mammalian subject such as a mouse or human, and the contacting step comprises administering the siRNA to the subject or comprises expressing the siRNA in the subject. According to certain embodiments of the invention the siRNA is expressed inducibly and/or in a cell-type or tissue specific manner.

By “biological system” is meant any vessel, well, or container in which biomolecules (e.g., nucleic acids, polypeptides, polysaccharides, lipids, and the like) are placed; a cell or population of cells; a tissue; an organ; an organism, and the like. Typically the biological system is a cell or population of cells, but the method can also be performed in a vessel using purified or recombinant proteins.

The invention provides siRNA molecules targeted to a transcript encoding any Chlamydia protease, e.g., tail specific protease CT441 or tail-specific protease Cpn0555. In particular, the invention provides siRNA molecules selectively or specifically targeted to a transcript encoding a polymorphic variant of such a transcript, wherein existence of the polymorphic variant in a subject is indicative of susceptibility to or presence of a Chlamydia infectious disease, or susceptibility to or presence of Chlamydia serovars which can cause sexually transmitted disease, ocular disease, blindness or atherosclerosis. The terms “selectively” or “specifically targeted to”, in this context, are intended to indicate that the siRNA causes greater reduction in expression of the variant than of other variants (i.e., variants whose existence in a subject is not indicative of susceptibility to or presence of a Chlamydia infectious disease). The siRNA, or collections of siRNAs, may be provided in the form of kits with additional components as appropriate.

B. Short Hairpin RNAs (shRNA)

RNA interference (RNAi), a mechanism of post-transcriptional gene silencing mediated by double-stranded RNA (dsRNA), is useful in a method for treating a Chlamydia infectious disease in a mammal by administering to the mammal a nucleic acid molecule (e.g., dsRNA) that hybridizes under stringent conditions to a Chlamydia protease target gene, and attenuates expression of said target gene. See Jain, Pharmacogenomics 5: 239-42, 2004 for a review of RNAi and siRNA. A further method of RNA interference in the present invention is the use of short hairpin RNAs (shRNA). A plasmid containing a DNA sequence encoding for a particular desired siRNA sequence is delivered into a target cell via transfection or virally-mediated infection. Once in the cell, the DNA sequence is continuously transcribed into RNA molecules that loop back on themselves and form hairpin structures through intramolecular base pairing. These hairpin structures, once processed by the cell, are equivalent to transfected siRNA molecules and are used by the cell to mediate RNAi of the desired protein. The use of shRNA has an advantage over siRNA transfection as the former can lead to stable, long-term inhibition of protein expression. Inhibition of protein expression by transfected siRNAs is a transient phenomenon that does not occur for times periods longer than several days. In some cases, this may be preferable and desired. In cases where longer periods of protein inhibition are necessary, shRNA mediated inhibition is preferable.

C. Full and Partial Length Antisense RNA Transcripts

Antisense RNA transcripts have a base sequence complementary to part or all of any other RNA transcript in the same cell. Such transcripts have been shown to modulate gene expression through a variety of mechanisms including the modulation of RNA splicing, the modulation of RNA transport and the modulation of the translation of mRNA (Denhardt, Ann N Y Acad. Sci. 660: 70, 1992; Nellen, Trends Biochem. Sci. 18: 419, 1993; Baker et al, Biochim. Biophys. Acta, 1489: 3, 1999; Xu, et al., Gene Therapy 7: 438, 2000; French et al., Curr. Opin. Microbiol. 3: 159, 2000; Terryn et al., Trends Plant Sci. 5: 1360, 2000).

D. Antisense RNA and DNA Oligonucleotides

Antisense nucleic acids are generally single-stranded nucleic acids (DNA, RNA, modified DNA, or modified RNA) complementary to a portion of a target nucleic acid (e.g., an mRNA transcript) and therefore able to bind to the target to form a duplex. Typically they are oligonucleotides that range from 15 to 35 nucleotides in length but may range from 10 up to approximately 50 nucleotides in length. Binding typically reduces or inhibits the function of the target nucleic acid. For example, antisense oligonucleotides may block transcription when bound to genomic DNA, inhibit translation when bound to mRNA, and/or lead to degradation of the nucleic acid. Reduction in expression of a Chlamydia protease polypeptide may be achieved by the administration of antisense nucleic acids or peptide nucleic acids comprising sequences complementary to those of the mRNA that encodes the polypeptide. Antisense technology and its applications are well known in the art and are described in Phillips, M. I. (ed.) Antisense Technology, Methods Enzymol., 2000, Volumes 313 and 314, Academic Press, San Diego, and references mentioned therein. See also Crooke, S. (ed.) “ANTISENSE DRUG TECHNOLOGY: PRINCIPLES, STRATEGIES, AND APPLICATIONS” (1^{st} Edition) Marcel Dekker; and references cited therein.

Antisense oligonucleotides can be synthesized with a base sequence that is complementary to a portion of any RNA transcript in the cell. Antisense oligonucleotides may modulate gene expression through a variety of mechanisms including the modulation of RNA splicing, the modulation of RNA transport and the modulation of the translation of mRNA (Denhardt, 1992). Various properties of antisense oligonucleotides including stability, toxicity, tissue distribution, and cellular uptake and binding affinity may be altered through chemical modifications including (i) replacement of the phosphodiester backbone (e.g., peptide nucleic acid, phosphorothioate oligonucleotides, and phosphoramidate oligonucleotides), (ii) modification of the sugar base (e.g., 2′-O-propylribose and 2′-methoxyethoxyribose), and (iii) modification of the nucleoside (e.g., C-5 propynyl U, C-5 thiazole U, and phenoxazine C) (Wagner, Nat. Medicine 1: 1116, 1995; Varga, et al., Immun. Lett. 69: 217, 1999; Neilsen, Curr. Opin. Biotech. 10: 71, 1999; Woolf, Nucleic Acids Res. 18: 1763, 1990).

The invention therefore provides a method of inhibiting expression of a gene encoding a Chlamydia protease comprising the step of (i) providing a biological system in which expression of a gene encoding Chlamydia protease is to be inhibited; and (ii) contacting the system with an antisense molecule targeted to a transcript encoding the Chlamydia protease. According to certain embodiments of the invention the Chlamydia protease is encoded by a gene within Chlamydia bacterial genome. In other embodiments, Chlamydia proteases are inhibited. The Chlamydia proteases include, but are not limited to, tail specific protease CT441 or tail-specific protease Cpn0555. According to certain embodiments of the invention the biological system comprises a cell, and the contacting step comprises expressing the siRNA in the cell. According to certain embodiments of the invention the biological system comprises a subject, e.g., a mammalian subject such as a mouse or human, and the contacting step comprises administering the siRNA to the subject or comprises expressing the siRNA in the subject. According to certain embodiments of the invention the siRNA is expressed inducibly and/or in a cell-type or tissue specific manner.

E. Ribozymes

Certain nucleic acid molecules referred to as ribozymes or deoxyribozymes have been shown to catalyze the sequence-specific cleavage of RNA molecules. The cleavage site is determined by complementary pairing of nucleotides in the RNA or DNA enzyme with nucleotides in the target RNA. Thus, RNA and DNA enzymes can be designed to cleave to any RNA molecule, thereby increasing its rate of degradation (Cotten et al, EMBO J. 8: 3861-3866, 1989; Usman et al., Nucl. Acids Mol. Biol. 10: 243, 1996; Usman, et al., Curr. Opin. Struct. Biol. 1: 527, 1996; Sun, et al., Pharmacol. Rev., 52: 325, 2000. See also e.g., Cotten et al, EMBO J. 8: 3861-3866, 1989).

The invention therefore provides a method of inhibiting expression of a gene encoding a Chlamydia protease comprising the step of (i) providing a biological system in which expression of a gene encoding Chlamydia protease is to be inhibited; and (ii) contacting the system with a ribozyme targeted to a transcript encoding the Chlamydia protease. According to certain embodiments of the invention the Chlamydia protease is encoded by a gene within Chlamydia bacterial genome. In other embodiments, Chlamydia proteases are inhibited. The Chlamydia proteases include, but are not limited to, tail specific protease CT441 or tail-specific protease Cpn0555. According to certain embodiments of the invention the biological system comprises a cell, and the contacting step comprises expressing the siRNA in the cell. According to certain embodiments of the invention the biological system comprises a subject, e.g., a mammalian subject such as a mouse or human, and the contacting step comprises administering the siRNA to the subject or comprises expressing the siRNA in the subject. According to certain embodiments of the invention the siRNA is expressed inducibly and/or in a cell-type or tissue specific manner.

### Therapeutic Applications

The small chemical molecule, siRNA molecule, dominant-negative mutants, or antibody inhibitors of Chlamydia protease identified by the methods of the present invention can be used in a variety of methods of treatment. Thus, the present invention provides compositions and methods for treating an Chlamydia infectious disease, including but not limited to, Chlamydia serovars which can cause sexually transmitted disease, ocular disease, blindness or atherosclerosis.

“Inflammation” or “inflammatory response” refers to an innate immune response that occurs when tissues are injured by bacteria, trauma, toxins, heat, or any other cause. The damaged tissue releases compounds including histamine, bradykinin, and serotonin. Inflammation refers to both acute responses (i.e., responses in which the inflammatory processes are active) and chronic responses (i.e., responses marked by slow progression and formation of new connective tissue). Acute and chronic inflammation can be distinguished by the cell types involved. Acute inflammation often involves polymorphonuclear neutrophils; whereas chronic inflammation is normally characterized by a lymphohistiocytic and/or granulomatous response. Inflammation includes reactions of both the specific and non-specific defense systems. A specific defense system reaction is a specific immune system reaction response to an antigen (possibly including an autoantigen). A non-specific defense system reaction is an inflammatory response mediated by leukocytes incapable of immunological memory. Such cells include granulocytes, macrophages, neutrophils and eosinophils. Examples of specific types of inflammation are diffuse inflammation, focal inflammation, croupous inflammation, interstitial inflammation, obliterative inflammation, parenchymatous inflammation, reactive inflammation, specific inflammation, toxic inflammation and traumatic inflammation.

C. trachomatis is the causative agent of trachoma, oculogential disease, infant pneumonia and lymphogranuloma venereum (LGV). C. trachomatis has a limited host range and only infects human epithelial cells (one strain can infect mice). The species is divided into three biovars (biological variants): trachoma, LGV and mouse pneumonitis. The human biovars have been further subdivided in to several serovars (serological variants; equivalent to serotypes) that differ in their major outer membrane proteins and which are associated with different diseases. Serovars A, B, Ba, and C cause trachoma prevalent in Asia and Africa. Serovars D through K cause eye disease, conjunctivitis, sexually transmitted disease, urethritis, cervicitis, and respiratory disease such as infant pneumonia. Serovars LGV1, LGV2, and LGV3 cause lymphogranuloma venerium. C. pneumoniae cause upper respiratory tract infection. C. pneumoniae is also associated with atherosclerosis. C. muridarum, (MoPn) is thought to be a mouse strain.

“Immune response” refers to the concerted action of lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues.

Preferably, treatment using Chlamydia protease inhibitors, e.g., a small chemical molecule inhibitor, a polypeptide inhibitor, or a peptidomimetic inhibitor of bacterial or parasite replication of the present invention could either be by administering an effective amount of the small chemical molecule inhibitor, the polypeptide inhibitor, or the peptidomimetic inhibitor to the patient, or by removing cells from the patient, supplying the cells with a polynucleotide of the present invention, and returning the engineered cells to the patient (ex vivo therapy). Moreover, the polypeptide or peptidomimetic of the present invention can be used as an antigen in a vaccine to raise an immune response against Chlamydia infectious disease.

### Pharmaceutical Compositions

Small molecule chemical inhibitors, siRNA inhibitors, or dominant negative mutants of Chlamydia protease activity, e.g., tail specific protease CT441 or tail-specific protease Cpn0555 protease activity, ligand mimetics, derivatives and analogs thereof, antibodies, or nucleic acid compositions, e.g., antisense oligonucleotides or double stranded RNA oligonucleotides (RNAi), or a dominant negative N terminal p40 fragment inhibitor (or analog thereof) of nuclear factor NF-κB activation prevents inflammation is useful in the present compositions and methods and can be administered to a human patient per se, in the form of a stereoisomer, prodrug, pharmaceutically acceptable salt, hydrate, solvate, acid salt hydrate, N-oxide or isomorphic crystalline form thereof, or in the form of a pharmaceutical composition where the compound is mixed with suitable carriers or excipient(s) in a therapeutically effective amount, for example, Chlamydia infectious disease, inflammatory disease, or cancer.

Pharmaceutically acceptable carriers are determined in part by the particular composition being administered, as well as by the particular method used to administer the composition. Accordingly, there is a wide variety of suitable formulations of pharmaceutical compositions for administering the antibody compositions (see, e.g., Remington's Pharmaceutical Sciences, Mack Publishing Co., Easton, Pa. 18^{th} ed., 1990, incorporated herein by reference). The pharmaceutical compositions generally comprise a differentially expressed protein, agonist or antagonist in a form suitable for administration to a patient. The pharmaceutical compositions are generally formulated as sterile, substantially isotonic and in full compliance with all Good Manufacturing Practice (GMP) regulations of the U.S. Food and Drug Administration.

### Labels

The particular label or detectable group used in the assay is not a critical aspect of the invention, so long as it does not significantly interfere with the specific binding of the small molecule chemical inhibitors or siRNA inhibitors of Chlamydia protease activity, e.g., tail specific protease CT441 or tail-specific protease Cpn0555 activity, ligand mimetics, derivatives and analogs thereof, antibodies, or nucleic acid compositions, e.g., antisense oligonucleotides or double stranded RNA oligonucleotides (RNAi), or a dominant negative N terminal p40 fragment inhibitor or analog thereof of nuclear factor NF-κB activation prevents inflammation used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g. Dynabeads™), fluorescent dyes (e.g., fluorescein isothiocyanate, Texas red, rhodamine, and the like), radiolabels (e.g., ³H, ¹⁴C, ³⁵S, ¹²⁵I, ¹²¹I, ¹²²In, ⁹⁹mTc), other imaging agents such as microbubbles (for ultrasound imaging), ¹⁸F, ¹¹C, ¹⁵O, (for Positron emission tomography), ^{99m}TC, ¹¹¹In (for Single photon emission tomography), enzymes (e.g., horse radish peroxidase, alkaline phosphatase and others commonly used in an ELISA), and calorimetric labels such as colloidal gold or colored glass or plastic (e.g. polystyrene, polypropylene, latex, and the like) beads. Patents that described the use of such labels include U.S. Pat. Nos. 3,817,837; 3,850,752; 3,939,350; 3,996,345; 4,277,437; 4,275,149; and 4,366,241, each incorporated herein by reference in their entirety and for all purposes. See also Handbook of Fluorescent Probes and Research Chemicals (6^{th} Ed., Molecular Probes, Inc., Eugene Oreg.).

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used. Where a ligand has a natural anti-ligand, for example, biotin, thyroxine, and cortisol, it can be used in conjunction with the labeled, naturally occurring anti-ligands. Alternatively, any haptenic or antigenic compound can be used in combination with an antibody.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidoreductases, particularly peroxidases. Fluorescent compounds include fluorescein and its derivatives, rhodamine and its derivatives, dansyl, umbelliferone, and the like Chemiluminescent compounds include luciferin, and 2,3-dihydrophthalazinediones, e.g., luminol. For a review of various labeling or signal producing systems which may be used, see, U.S. Pat. No. 4,391,904, incorporated herein by reference in its entirety and for all purposes.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple calorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

Frequently, the Chlamydia protease polypeptide, e.g., tail-specific protease CT441 or tail-specific protease Cpn0555 will be labeled by joining, either covalently or non-covalently, a substance which provides for a detectable signal.

### Treatment Regimes

The invention provides pharmaceutical compositions comprising one or a combination of small molecule chemical inhibitors, siRNA inhibitors, or dominant-negative mutants of Chlamydia protease activity, e.g., tail specific protease CT441 or tail-specific protease Cpn0555 activity, monoclonal, polyclonal or single chain Fv; intact or binding fragments thereof, or nucleic acid compositions, e.g., antisense oligonucleotides, double stranded RNA oligonucleotides (RNAi) or DNA oligonucleotides (vectors) containing nucleotide sequences encoding for the transcription of shRNA molecules, or dominant negative N terminal p40 peptide fragment inhibitor or analog thereof of nuclear factor NF-κB activation which prevents inflammation formulated together with a pharmaceutically acceptable carrier. Some compositions include a combination of multiple (e.g., two or more) small chemical molecules, siRNA molecules, monoclonal antibodies or antigen-binding portions thereof of the invention. In some compositions, each of the antibodies or antigen-binding portions thereof of the composition is a monoclonal antibody or a human sequence antibody that binds to a distinct, pre-selected epitope of an antigen.

In prophylactic applications, pharmaceutical compositions or medicaments are administered to a patient susceptible to, or otherwise at risk of a disease or condition (i.e., an immune disease) in an amount sufficient to eliminate or reduce the risk, lessen the severity, or delay the outset of the disease, including biochemical, histologic and/or behavioral symptoms of the disease, its complications and intermediate pathological phenotypes presenting during development of the disease. In therapeutic applications, compositions or medicants are administered to a patient suspected of, or already suffering from such a disease in an amount sufficient to cure, or at least partially arrest, the symptoms of the disease (biochemical, histologic and/or behavioral), including its complications and intermediate pathological phenotypes in development of the disease. An amount adequate to accomplish therapeutic or prophylactic treatment is defined as a therapeutically- or prophylactically-effective dose. In both prophylactic and therapeutic regimes, agents are usually administered in several dosages until a sufficient immune response has been achieved. Typically, the immune response is monitored and repeated dosages are given if the immune response starts to wane.

### Effective Dosages

Effective doses of the small molecule chemical inhibitors, siRNA inhibitors, or dominant-negative mutants of Chlamydia protease activity e.g., tail specific protease CT441 or tail-specific protease Cpn0555 activity, or nucleic acid compositions, e.g., antisense oligonucleotides, double stranded RNA oligonucleotides (RNAi), or DNA oligonucleotides (vectors) containing nucleotide sequences encoding for the transcription of shRNA molecules, or a dominant negative N terminal p40 peptide fragment inhibitor or analog thereof of nuclear factor NF-κB activation prevents inflammation is useful for the treatment of Chlamydia infectious disease described herein vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. Treatment dosages need to be titrated to optimize safety and efficacy.

For administration with a small chemical molecule, nucleic acid, siRNA, or antibody composition, the dosage ranges from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once per every two weeks or once a month or once every 3 to 6 months. In some methods, two or more small chemical molecules or siRNA molecules with different binding specificities are administered simultaneously, in which case the dosage of each small chemical molecule, siRNA molecule, or antibody administered falls within the ranges indicated. Small chemical molecule, siRNA molecule, or antibody is usually administered on multiple occasions. Intervals between single dosages can be weekly, monthly or yearly. Intervals can also be irregular as indicated by measuring blood levels of small chemical molecule, siRNA molecule, or antibody in the patient. In some methods, dosage is adjusted to achieve an antibody concentration of 1-1000 μg/ml and in some methods 25-300 μg/ml. Alternatively, antibody can be administered as a sustained release formulation, in which case less frequent administration is required. Dosage and frequency vary depending on the half-life of the compound in the patient. In general, human antibodies show the longest half life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

Doses for small chemical molecules, siRNA molecules, or nucleic acids range from about 10 ng to 1 g, 100 ng to 100 mg, 1 μg to 10 mg, or 30-300 μg DNA per patient. Doses for infectious viral vectors vary from 10-100, or more, virions per dose.

### Prodrugs

The present invention is also related to prodrugs of the agents obtained by the methods disclosed herein. Prodrugs are agents which are converted in vivo to active forms (see, e.g., R. B. Silverman, 1992, The Organic Chemistry of Drug Design and Drug Action, Academic Press, Chp. 8). Prodrugs can be used to alter the biodistribution (e.g., to allow agents which would not typically enter the reactive site of the protease) or the pharmacokinetics for a particular agent. For example, a carboxylic acid group, can be esterified, e.g., with a methyl group or an ethyl group to yield an ester. When the ester is administered to a subject, the ester is cleaved, enzymatically or non-enzymatically, reductively, oxidatively, or hydrolytically, to reveal the anionic group. An anionic group can be esterified with moieties (e.g., acyloxymethyl esters) which are cleaved to reveal an intermediate agent which subsequently decomposes to yield the active agent. The prodrug moieties may be metabolized in vivo by esterases or by other mechanisms to carboxylic acids.

Examples of prodrugs and their uses are well known in the art (see, e.g., Berge et al., “Pharmaceutical Salts”, J. Pharm. Sci. 66:1-19, 1977). The prodrugs can be prepared in situ during the final isolation and purification of the agents, or by separately reacting the purified agent in its free acid form with a suitable derivatizing agent. Carboxylic acids can be converted into esters via treatment with an alcohol in the presence of a catalyst.

Examples of cleavable carboxylic acid prodrug moieties include substituted and unsubstituted, branched or unbranched lower alkyl ester moieties, (e.g., ethyl esters, propyl esters, butyl esters, pentyl esters, cyclopentyl esters, hexyl esters, cyclohexyl esters), lower alkenyl esters, dilower alkyl-amino lower-alkyl esters (e.g., dimethylaminoethyl ester), acylamino lower alkyl esters, acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, dilower alkyl amides, and hydroxy amides.

### Routes of Administration

Small chemical molecule, siRNA molecule, or antibody compositions for treatment or amelioration of Chlamydia infectious disease, or nucleic acid compositions, e.g., antisense oligonucleotides, double stranded RNA oligonucleotides (RNAi), or DNA oligonucleotides (vectors) containing nucleotide sequences encoding for the transcription of shRNA molecules, for the treatment of Chlamydia infectious disease, or dominant negative N terminal p40 peptide fragment inhibitor or analog thereof of nuclear factor NF-κB activation to prevent inflammation can be administered by parenteral, topical, intravenous, oral, subcutaneous, intraarterial, intracranial, intraperitoneal, intranasal or intramuscular means for prophylactic as inhalants for small chemical molecule, siRNA molecule or antibody preparations targeting chlamydial infectious disease, and/or therapeutic treatment. The most typical route of administration of an immunogenic agent is subcutaneous although other routes can be equally effective. The next most common route is intramuscular injection. This type of injection is most typically performed in the arm or leg muscles. In some methods, agents are injected directly into a particular tissue where a tumor is found, for example intracranial injection or convection enhanced delivery. Intramuscular injection or intravenous infusion are preferred for administration of antibody. In some methods, particular therapeutic antibodies are delivered directly into the cranium. In some methods, antibodies are administered as a sustained release composition or device, such as a Medipad™ device.

Agents of the invention can optionally be administered in combination with other agents that are at least partly effective in treating various diseases including various immune-related diseases. In the case of infection in the brain, agents of the invention can also be administered in conjunction with other agents that increase passage of the agents of the invention across the blood-brain barrier (BBB).

### Formulation

Small chemical molecule, siRNA molecule, or antibody inhibitors of Chlamydia protease, nucleic acid compositions, e.g., antisense oligonucleotides, double stranded RNA oligonucleotides (RNAi), or DNA oligonucleotides (vectors) containing nucleotide sequences encoding for the transcription of shRNA molecules, for the treatment of Chlamydia infectious disease, or a dominant negative N terminal p40 peptide fragment inhibitor or analog thereof of nuclear factor NF-κB activation to prevent inflammation are often administered as pharmaceutical compositions comprising an active therapeutic agent, i.e., and a variety of other pharmaceutically acceptable components. See Remington's Pharmaceutical Science (15^{th} ed., Mack Publishing Company, Easton, Pa., 1980). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

For parenteral administration, compositions of the invention can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies can be administered in the form of a depot injection or implant preparation which can be formulated in such a manner as to permit a sustained release of the active ingredient. An exemplary composition comprises monoclonal antibody at 5 mg/mL, formulated in aqueous buffer consisting of 50 mM L-histidine, 150 mM NaCl, adjusted to pH 6.0 with HCl.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins. Glenn et al., Nature 391: 851, 1998. Co-administration can be achieved by using the components as a mixture or as linked molecules obtained by chemical crosslinking or expression as a fusion protein.

Alternatively, transdermal delivery can be achieved using a skin patch or using transferosomes. Paul et al., Eur. J. Immunol. 25: 3521-24, 1995; Cevc et al., Biochem. Biophys. Acta 1368: 201-15, 1998.

The pharmaceutical compositions are generally formulated as sterile, substantially isotonic and in full compliance with all Good Manufacturing Practice (GMP) regulations of the U.S. Food and Drug Administration.

### Toxicity

Preferably, a therapeutically effective dose of the small chemical molecule, siRNA molecule, antibody, or nucleic acid compositions, e.g., antisense oligonucleotides, double stranded RNA oligonucleotides (RNAi), or DNA oligonucleotides (vectors) containing nucleotide sequences encoding for the transcription of shRNA molecules, or dominant negative N terminal p40 peptide fragment inhibitor or analog thereof of nuclear factor NF-κB activation prevents inflammation as described herein will provide therapeutic benefit without causing substantial toxicity.

Toxicity of the proteins described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1,

### Kits

Also within the scope of the invention are kits comprising the small chemical molecule, siRNA molecule, antibody, or nucleic acid compositions, e.g., antisense oligonucleotides, double stranded RNA oligonucleotides (RNAi), or DNA oligonucleotides (vectors) containing nucleotide sequences encoding for the transcription of shRNA molecules), or dominant negative N terminal p40 peptide fragment inhibitor or analog thereof of nuclear factor NF-κB activation prevents inflammation, of the invention and instructions for use. The kit can further contain a least one additional reagent, or one or more additional human antibodies of the invention (e.g., a human antibody having a complementary activity which binds to an epitope in the antigen distinct from the first human antibody). Kits typically include a label indicating the intended use of the contents of the kit. The term label includes any writing, or recorded material supplied on or with the kit, or which otherwise accompanies the kit.

Other embodiments and uses will be apparent to one skilled in the art in light of the present disclosures.

The invention will be further described with reference to the following examples; however, it is to be understood that the invention is not limited to such examples.

### EXEMPLARY EMBODIMENTS

### Example 1

### Chlamydia Targeted NF-κB Pathway

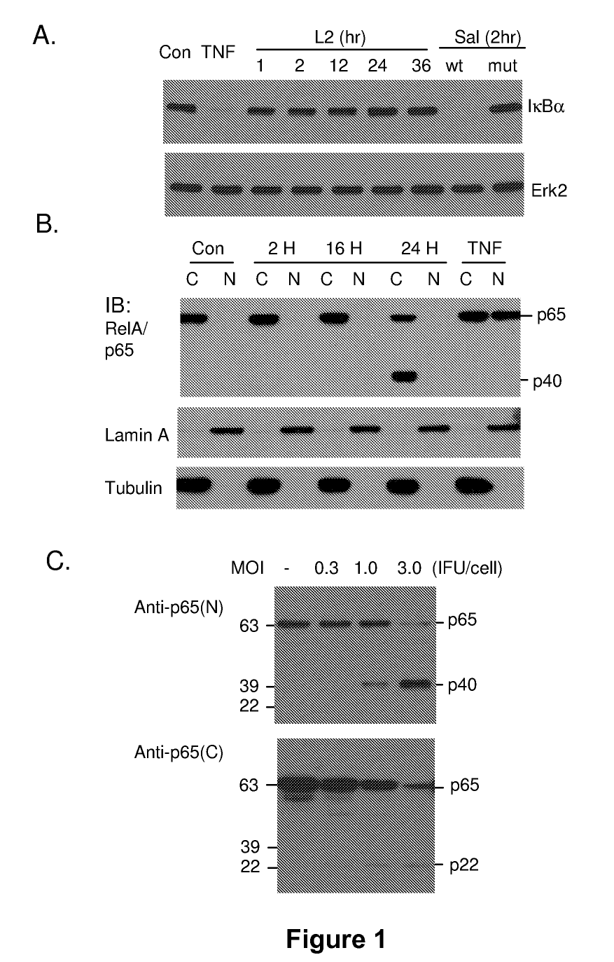

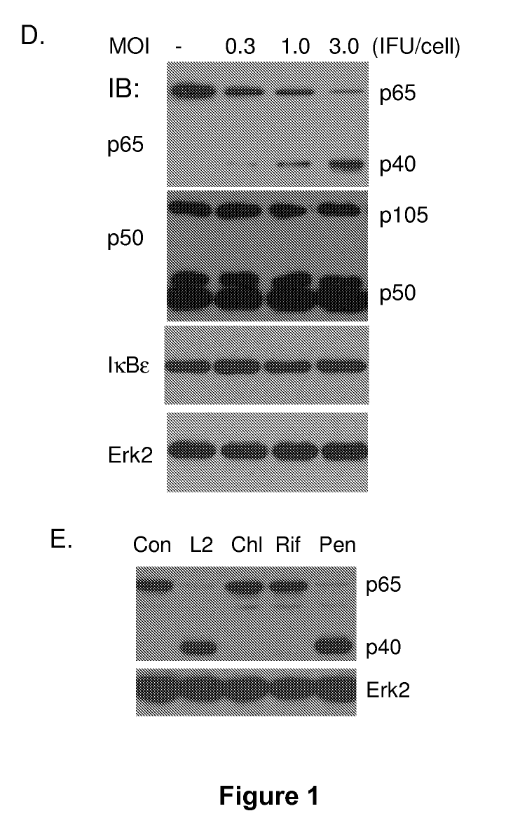

The host inflammatory response to bacterial infection or proinflammatory cytokine and LPS stimulation is mediated by NF-κB activity, which is regulated by the selective degradation of IκB proteins. Karin and Ben-Neriah, Annu. Rev. Immunol 18: 621, 2000; Richmond, Nat. Rev. Immunol 2: 664, 2002. To investigate whether Chlamydia targeted the NF-κB pathway to interfere with host inflammatory response, we first examined the integrity of IκBα, a regulatory molecule of NF-κB activation. To this end, monolayers of Hela 229 cells were infected with C. trachomatis LGV2 at 1 inclusion forming unit per cell (1 IFU/cell) for varying time points. Lad et al., J Immunol 174: 7186, 2005. As controls, the cells were infected with invasive or noninvasive Salmonella typhimurium for 2 hr or treated with TNFα to induce IκBα degradation. Jones et al., J Exp Med 180: 15, 1994; Neish et al., Science 289: 1560, 2000. Unlike TNFα treatment or infection with pathogenic Salmonella typhimurium which caused IκBα degradation, infection with C. trachomatis did not affect IκBα expression nor its degradation (FIG. 1A), suggesting that Chlamydia infection does not promote NF-κB activation.

To verify these findings, we examined the nuclear translocation of p65 as a measurement of NF-κB activation. The NF-κB consists of a heterodimeric complex composed of two subunits, commonly p50/NF-κB1 and p65/RelA, which are sequestered in the cytoplasm and rendered inactive through their association with inhibitory molecules, including IκBα. The distribution of p65 in the cytosol and the nuclei of infected and uninfected control cells was detected after fractionation by immunoblotting analysis with an N-terminus specific anti-p65 antibody. Joseph and Moll, Methods Mol. Biol. 234: 211, 2003. Consistent with a previous study (Molestina et al., Infect. Immun. 68: 4282, 2000) and our results from the IκBα degradation studies, Chlamydia infection did not promote NF-κB activation as determined by the absence of p65 nuclear translocation (FIG. 1B). However, a protein band of approximately 40 kDa was detected from the cytosolic preparation of cells infected for 24 hr, suggesting that Chlamydia infection can potentially cause p65 cleavage.

The cleavage of p65 was confirmed by the detection of a 22 kDa fragment with a C-terminus specific antibody against p65 (FIG. 1C). Enhanced detection of p22 was observed in cells treated with MG-132 (data not shown), suggesting that this cleavage product was subjected to proteasomal degradation. The 40 kDa fragment was further characterized with MALDI proteomics. Thus, 293T cells, transfected for GST-p65 protein expression, were infected with LGV2 for 28 hr. After purification, the cleavage product was subjected to trypsin digestion followed by MALDI TOF identification. Seven proteolytic fragments were detected, which covered the N-terminal GST tag and the RHD region of p65 (supplemental data). Together, these results indicate that Chlamydia infection leads to p65 cleavage into a p22 fragment of the C-terminus and a p40 fragment which contains the RHD domain of DNA binding.

The cleavage of p65 was selective since expression of p50/105, IκBα and IκBε proteins of the NF-κB signaling pathway was not affected by Chlamydia infection (FIG. 1D). In addition, no p65 cleavage was detected in Chlamydia-infected murine cells (data not shown). Furthermore, the cleavage activity was dependent on bacterial growth since Chlamydia cleavage of p65 was blocked by bacteria-static chloramphenicol or rifampin (FIG. 1E), but not by penicillin G, an antibiotic that inhibits Chlamydia maturation but not its growth. Moulder, Infect Agents Dis 2: 87, 1993.

### Example 2

### Role for Chlamydia Protease in Reduced Inflammatory Response to Infection

FIG. 1 shows Chlamydia trachomatis infection does not induce IκBα degradation, but promotes the cleavage of p65/RelA. A. Hela 229 cells were infected with Chlamydia trachomatis LGV2 at 1 IFU/cell for varying times, or with invasive (wt) or invasion-defective (mut) Salmonella typhimurium for 2 hr. IκBα expression and degradation were detected by immunoblotting analyses. TNFα (10 ng/ml) was used as a positive control. B. Hela 229 cells were infected with LGV2 at 1 IFU/cell for 2, 16 and 24 hr. The cytosolic (C) and nuclear (N) fractions were separated and redistribution of p65/RelA was detected by immunoblotting analysis. The expression of lamin A and β-tubulin were used as loading controls for nuclear and cytosolic fractions, respectively. C. Whole cell lysates from Chlamydia-infected cells were separated by SDS-PAGE and cleavage of p65 was detected with an N- or C-terminus specific antibody against p65. D. Selective cleavage of p65 by Chlamydia infection. The expression of NF-κB pathway signaling molecules in Chlamydia-infected and non-infected control cells was determined by immunoblotting analysis. E. Inhibition of Chlamydia growth blocks p65 cleavage. The infected cells were treated with rifampin (Rif, 0.1 μg/ml) or chloramphenicol (Chl, 60 μg/ml), reagents that inhibit Chlamydia growth by blocking transcription or translation, respectively. Penicillin G (Pen, 100 μg/ml) that does not block Chlamydia replication but its maturation was included as a control. The expression of β-tubulin or Erk2/MAPK was used as loading controls. All experiments were performed at least 3 times.

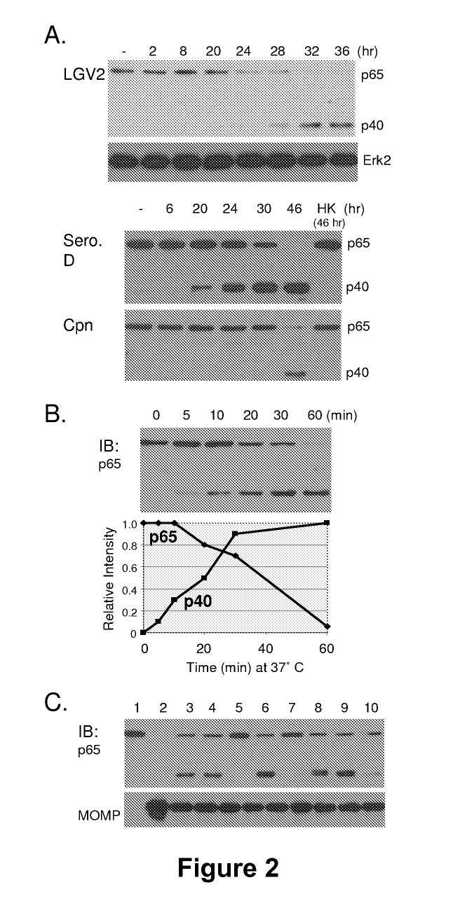

Chlamydia trachomatis has a unique biphasic developmental cycle. Moulder, Microbiol. Rev. 55: 143, 1991; Stephens, Infect. Agents Dis. 1: 279, 1992. Following internalization, the infectious elementary body (EB) differentiates into a metabolically active reticulate body (RB) for replication within 8-12 hr p.i. The RB begins to redifferentiate into EB about 20-24 hr p.i. No p65 cleavage was detected during the Chlamydia replication phase (FIG. 2A). The cleavage was detected at approximately 24 hr p.i. and persisted thereafter. In fact, serovar D exhibited a similar pattern of p65 cleavage, while cleavage by C. pneumoniae was delayed but was consistent with the slower growth rate of this organism, suggesting that the cleavage activity was associated with EB. This conclusion was substantiated by results from in vitro protease activity assays. Gradient-purified EB was solublized with a buffer containing 1% NP-40. The fraction containing solublized bacterial proteins was found to have p65 cleavage activity (FIG. 2B). In addition, the activity was independent of multivalent cations since inclusion of EDTA had no effect on p65 cleavage. Instead, lactacystin inhibited p65 cleavage activity of Chlamydia (FIG. 2C), suggesting potential involvement of serine proteases. Fenteany et al., Science 268: 726, 1995.

FIG. 2 shows the p65 cleavage activity is associated with the chlamydial elementary body (EB). A. Time course of p65 cleavage during Chlamydia infection. Monolayers of Hela 229 cells were infected with Chlamydia sp. at 1 IFU/cell for varying times. Protein expression and cleavage of p65 were detected by immunoblotting analyses. The p65 cleavage activity was detected at the late stages of Chlamydia growth. B. Soluble proteins from gradient-purified EB have p65 cleavage activity. Purified Chlamydia EB was solubilized with a buffer containing 1% NP-40. One microgram of bacterial proteins was incubated with p65 from 293T cells at 37° C. for varying times as indicated. The graph represents the rates of p65 disappearance and p40 appearance in the same reaction. C. Cleavage of p65 was blocked by serine-protease inhibitors. In parallel experiments, protease inhibitors were included in the in vitro p65 cleavage assay. Lane 1: 293T lysate; Lane 2: solubilized EB; Lane 3: p65 and EB proteins incubated at 37° C. for 60 min. Lane 4: Roche protease inhibitor cocktail at 10×; Lane 5: heat-inactivated EB; Lane 6: EDTA at 5 mM; Lane 7: Lactacystin at 10 μM; Lane 8: MG132 at 20 μM; Lane 9: TPCK at 20 μM; and Lane 10: TLCK at 20 μM. The presence of the chlamydial major outer membrane protein (MOMP) was used as a measure of EB protein input (lower panel).

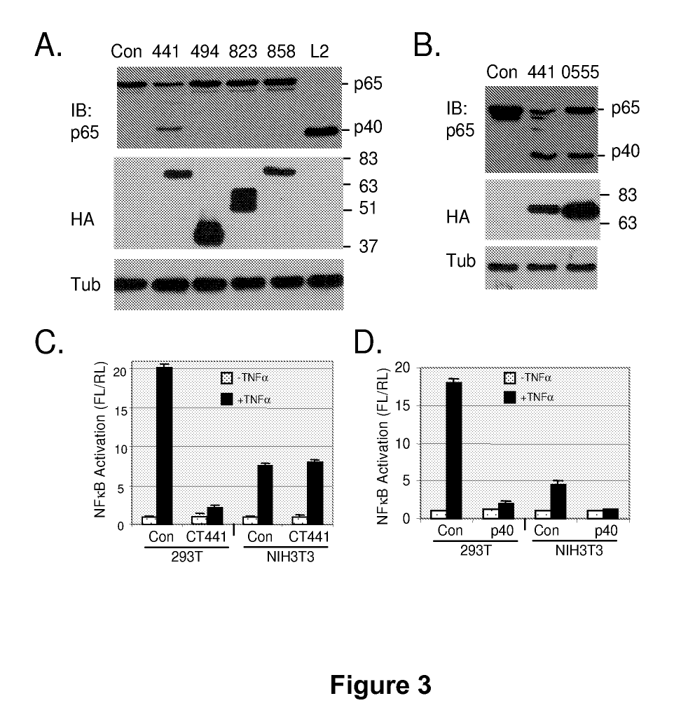

The chlamydial genome encodes 16 proteases (Stephens et al., Science 282: 754, 1998), including four putative proteases that contain a leader sequence for periplasmic secretion and potential translocation by the chlamydial type III secretory system. The mature peptides of those putative proteases were therefore screened for their ability to cleave p65 after expression in 293T cells. As shown in FIG. 3A, expression of CT441 caused p65 cleavage. CT441 encodes a tail-specific protease (Tsp) that is well conserved among Chlamydia species. Indeed, the protein from serovar A of ocular infection is identical to CT441 of serovar D, and the corresponding Tsp (Cpn0555) of C. pneumoniae also displayed p65 cleavage activity (FIG. 3B). Although CPAF (CT858), a chlamydial protease detected during an infection but not in purified EB (Zhong et al., J. Exp. Med. 193: 935, 2001), was reported to degrade the RFX transcriptional factor (Zhong et al., J. Exp. Med. 191: 1525, 2000), CT858 did not exhibit p65 cleavage activity.

It was hypothesized that Chlamydiae utilize the Tsp to regulate NF-κB activity and hence host inflammatory response. The lack of genetic mutants, or tools to generate such mutants, prevented a direct demonstration of CT441 as a virulence factor in an infection setting. We therefore investigated whether expression of CT441 interfered with NF-κB signal transduction. To this end, 293T cells were transfected with pIL8-luciferase (Kim et al., Cancer Res 65: 8784, 2005) and a plasmid for CT441 expression. The cells were then treated with TNFα to induce luciferase expression. In parallel experiments, we also included a mouse cell line for control purposes since murine p65 is resistant to cleavage by Chlamydia infection. As anticipated, expression of CT441 in 293T cells inhibited TNFα-induced luciferase activity. In contrast, CT441 showed no effect on TNFα-induced luciferase expression in NIH3T3 cells (FIG. 3C). These data suggested that the inhibition of NF-κB activity was associated with CT441 cleavage of human p65. The p40 cleavage product (amino acid residues 1-351) was then investigated for its inhibitory effect on TNFα-induced NF-κB activation. Expression of p40, which contains an intact RHD domain for DNA binding and NLS for nuclear translocation, inhibited luciferase expression (FIG. 3D). Together, these data suggest that CT441 interferes with NF-κB signal transduction to regulate host inflammatory response.

P65/RelA (Homo sapiens) has the following amino acid sequence. GenBank Accession Number NM 021975 [gi46430498];

SEQ ID NO:1
MDELFPLIFP AEPAQASGPY VEIIEQPKQR GMRFRYKCEG RSAGSIPGER STDTTKTHPT IKINGYTGPG
TVRISLVTKD PPHRPHPHEL VGKDCRDGFY EAELCPDRCI HSFQNLGIQC VKKRDLEQAI SQRIQTNNNP
FQVPIEEQRG DYDLNAVRLC FQVTVRDPSG RPLRLPPVLP HPIFDNRAPN TAELKICRVN RNSSSCLGGD
EIFLLCDKVQ KEDIEVYFTG PSWEARGSFS QADVHRQVAI VFRTPPYADP SLQAPVRVSM QLRRPSDREL
SEPMEFQYLP DTDDRHRIEE KRKRTYETFK SIMKKSPFSG PTDPRPPPRR IAVPSRSSAS VPKPAPQPYP
FTSSLSTINY DEFPTMVFPS GQISQASALA PAPPQVLPQA PAPAPAPAMV SALAQAPAPV PVLAPGPPQA
VAPPAPKPTQ AGEGTLSEAL LQLQFDDEDL GALLGNSTDP AVFTDLASVD NSEFQQLLNQ GIPVAPHTTE
PMLMEYPEAI TRLVTGAQRP PDPAPAPLGA PSLPNGLLSG DEDFSSIADM DFSALLSQIS S.

Chlamydia trachomatis tail specific protease has the following amino acid sequence. GenBank Accession Number AAC68040 [gi:3328872]; Stephens et al., Science 282: 754-759, 1998.

| SEQ ID NO:2 | |
|---|---|
| 1 | mmrfarfcll vltlfpqlaf saeplrrqdv rktvdklveh hidtqqispy ilsrsledyv |
| 61 | rsfdshkayl tqdevfshaf seeatrplfk qyqednfssf keldtciqqs israrewrss |
| 121 | wltdsirviq damshtiekk psawassiee vkqrqydlll syasiyleda aknryqgkeh |
| 181 | alvklcirqi enhenpyigi ndhgyrmspe eeansfhvri iksiahslda htayfsqeea |
| 241 | lsmraqlekg mcgigvvlke didgvvvkev laggpadktg slrvgdiiyr vngknientp |
| 301 | fpgvldslrg spgssvtldi hrqnndhviq lrrekillds rrvdvsyepy gngiigkitl |
| 361 | hsfyegenqv sseqdlrkai relqeknllg lvldirentg gflsqaikvs glfltngvvv |
| 421 | vsryadgsvk ryrtispqkf ydgplavlvs kssasaaeiv aqtlqdygva livgdqqtyg |
| 481 | kgtiqhqtit gsnsqedffk vtvgryysps gkstqlegvk sdivipsrya edklgerfle |
| 541 | yalpadqyen vindnlgdld inirpwfqky ysphlqkpel vwremlpqla hnsqerlekn |
| 601 | knfeifvqhl kktnkqdrsf gsndlqmees vnivkdmill ksis. |

FIG. 3 shows the identification of the chlamydial protease responsible for the cleavage of p65 protein. A. Putative chlamydial proteases were expressed as N-terminal HA-tagged proteins in 293T cells and screened for their ability to cleave p65. Expression of CT441 of Chlamydia trachomatis, a tail specific protease, resulted in selective cleavage of p65. 441, 494, 823 and 858 represent chlamydial CT441, CT494, CT823, and CT858, respectively. B. Cpn0555, an ortholog of CT441 from Chlamydia pneumoniae, also displayed p65 cleavage activity. C, D. Expression of CT441 or p40, a cleavage product derived from the N-terminus of p65, inhibited NF-κB activity. Monolayers of 293T or NIH3T3 cells were co-transfected with pIL8-Luc (Kim et al., Cancer Res 65: 8784, 2005) and a plasmid for the expression of chlamydial protease CT441 or the p40 cleavage product of p65. pSV40-RL was included as an internal control for transfection reactions. The cells were treated with TNFα (10 ng/ml) 18 hr post transfection to induce luciferase expression. Luciferase activities were determined with a Dual-Glo reagent kit (Promega) 18 hr after TNFα treatment. The ratio of firefly and Renilla luciferase activities (FL/RL) was plotted. All transfections were performed in duplicate and the data are presented as means +/− error.

Chlamydia infection is the most common cause of the notifiable diseases in the United States. Centers for Disease Control and Prevention, MMWR Morb. Mortal. Wkly. Rep. 52: 16, 2003. Most patients are not aware of an infection since the disease generally remains asymptomatic. Chlamydiae are obligate intracellular bacterial pathogens which have evolved through sophisticated mechanisms to secure a favorable habitat for progeny production while avoiding harm to their host. Chlamydiae are the only bacteria with sequenced genomes that encode a protease which is homologous to A20 of protein ubiquitination/deubiquitination activity. Makarova et al., Trends Biochem. Sci. 25: 50, 2000; Wertz et al., Nature 430: 694, 2004; Boone et al., Nat Immunol 5: 1052, 2004. We found that Chlamydiae also contain a tail-specific protease that selectively cleaves the p65/RelA subunit of NF-κB to potentially interfere with host inflammatory response. In addition, several limited examples demonstrate that viral and bacterial pathogens can exploit the NF-κB pathway to regulate cellular response. Santoro et al, EMBO J 22: 2552, 2003; Tato and Hunter, Infect. Immun. 70: 3311, 2002; Boyer and Lemichez, Nat Rev Micro 2: 779, 2004. Nonpathogenic Salmonella sp. was found to inhibit NF-κB translocation by blocking ubiquitination of IκBα (Neish et al., Science 289: 1560, 2000) and a commensal anaerobic bacterium to attenuate inflammation by causing efflux of nuclear p65 (Kelly et al., Nat. Immunol 5: 104, 2004). Together, these studies define novel molecular mechanisms which are utilized by microorganisms to evade the immune system.

### Example 3

### Materials and Methods

Bacterial strains. Chlamydia trachomatis serovar C (TW-3), D (UW-3/Cx), F (IC-Cal-3) and C. pneumoniae CWL-029 were obtained from ATCC. The bacteria were propagated in Hela 229 cells in the presence of cycloheximide and gradient purified. Caldwell et al., Infect Immun. 31: 1161, 1981. C. trachomatis lymphogranuloma venereum (LGV2) was obtained from G. Zhong and was propagated in Hela 229 cells without the use of cycloheximide. The titers of these bacteria were determined in Hela 229 cells and were expressed as inclusion forming units (IFU) per milliliter.

Infection assays. Monolayers of Hela 229 cells, or cells as specified, were infected with LGV2 at 1 IFU/cell or as otherwise stated. To improve infection efficiency by serovars or biovars other than LGV2, the cells were treated with 30 μg/ml DEAE-dextran prior to inoculation. No cycloheximide was used during infection assays.

Western blots. Except as noted, extracts were prepared by lysis of cells with a buffer containing 143 mM NaCl, 50 mM Tris-HCl (pH 7.5), 1 mM DTT, 1% NP-40, 0.1% SDS and protease inhibitors (Roche). Soluble proteins were separated by SDS-PAGE or Tris-Glycine gels (Novex). Blots were probed with specific antibodies. Horseradish peroxidase conjugated secondary antibodies (Sigma) and ECL reagent kit (Pierce) were used for detection.

In vitro p65 cleavage assays. Gradient purified chlamydial EB were solubilized with Tris-buffered saline, supplemented with 1% NP-40. The soluble proteins were collected and used for p65 cleavage assays. To assay for p65 cleavage ability, 1 μg bacterial proteins were resuspended in 30 μl reaction buffer containing 20 mM Tris-HCl, 143 mM NaCl, with or without p65 protein expressed in 293T cells or the endogenous p65 from 293T cell lysate. For inhibition assays, an inhibitor was added 30 min prior to the addition of p65 protein. All assays were performed at 37° C. for 60 min or as otherwise indicated.

Constructs for expression of bacterial proteases. cDNAs of the putative proteases (Stephens et al., Science 282: 754, 1998) were PCR amplified using purified bacteria as templates and were inserted into pRK5/HA for N-terminal HA-tagged protein expression. The proteins were expressed in 293T cells by transient transfection using Fugene-6 (Roche) and assayed for their ability to cleave the endogenous p65 protein with Western blotting analyses.

Reporter gene assay. Monolayers of 293T or NIH3T3 cells in 24-well plates were transfected in duplicates with a mixture of DNA preparations (pIL8-GL, 0.06 μg; pSV40-RL, 0.01 μg; and 0.13 μg pRK5/Myc or a plasmid DNA for protein expression). To construct a plasmid for p40 expression, we first mapped the cleavage site of p65 to amino acid residue 351 with standard molecular biology protocols. The DNA corresponding to aa 1-351 was inserted into pRK5/Myc for Myc-p40 protein expression. The transfected cells were treated with human TNFα (10 ng/ml) 18 hr post transfection to induce firefly luciferase expression or remained untreated. The cells were harvested 18 hr later and luciferase activities were assayed with the Dual-Glo reagent kit (Promega). The results were presented as the ratio of firefly luciferase activity against renilla luciferase activity (mean +/− error of duplicate samples), using 1 as a ratio for the corresponding controls.

All publications and patent applications cited in this specification are herein incorporated by reference in their entirety for all purposes as if each individual publication or patent application were specifically and individually indicated to be incorporated by reference for all purposes.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

What is claimed:

1. A method for treating inflammation or inflammatory disease in a mammalian subject comprising,
administering an N-terminal p40 fragment of p65/RelA or an analog thereof to the mammalian subject, wherein the p40 fragment or analog thereof is administered in an amount effective to reduce or eliminate the inflammation or inflammatory disease or to prevent its occurrence or recurrence.

2. The method of claim 1 wherein the p40 fragment is a dominant-negative molecule, dominant-negative peptide or dominant-negative peptidomimetic.

3. The method of claim 1 wherein the p40 fragment or analog thereof is a small chemical molecule, monoclonal antibody, polyclonal antibody, peptide, peptidomimetic, or a nucleic acid.

4. The method of claim 3 wherein the nucleic acid encodes the p40 fragment or the analog thereof.

5. The method of claim 2 wherein the p40 fragment or analog thereof has at least 95% sequence identity to amino acids 1 to 351 of SEQ ID NO:1.

6. A method for treating inflammation or inflammatory disease in a mammalian subject comprising,
administering a Chlamydia protease or an analog thereof to the mammalian subject, wherein the Chlamydia protease or analog thereof is administered in an amount effective to reduce or eliminate the inflammation or inflammatory disease or to prevent its occurrence or recurrence.

7. The method of claim 6 wherein the Chlamydia protease is a Chlamydia tail-specific protease.

8. The method of claim 7 wherein the Chlamydia protease or analog thereof has at least 95% sequence identity to SEQ ID NO:2.

9. The method of claim 6 wherein the Chlamydia protease is Chlamydia trachomatis tail-specific protease CT441, Chlamydia pneumoniae tail-specific protease Cpn0555, or Chlamydia muridarum tail-specific protease TC0725.

10. A method for treating persistent Chlamydia infection in a mammalian subject comprising,
administering an inhibitor of Chlamydia protease, wherein the inhibitor is administered in an amount effective to reduce or eliminate the persistent Chlamydia infection or to prevent its occurrence or recurrence.

11. The method of claim 10 wherein the Chlamydia protease is a Chlamydia tail specific protease.

12. The method of claim 10 wherein the Chlamydia protease is Chlamydia trachomatis tail specific protease CT441, Chlamydia pneumoniae tail-specific protease Cpn0555, or Chlamydia muridarum tail specific protease TC0725.

13. The method of claim 9 wherein the inhibitor is a small chemical compound, short interfering RNA, dominant-negative molecule, short hairpin RNA, ribozyme, antisense oligonucleotide, antibody, peptide or peptidomimetic.

14. A method for treating Chlamydia infection in a mammalian subject comprising,
administering a polypeptide fragment of Chlamydia protease to induce an immune response in the mammalian subject wherein the polypeptide fragment is administered in an amount effective to reduce or eliminate the Chlamydia infection or to prevent its occurrence or recurrence.

15. The method of claim 14 wherein the immune response is a cytotoxic T cell response or a humoral immune response.

16. The method of claim 14, further comprising administering an adenovirus vector encoding the polypeptide fragment of Chlamydia protease.

17. The method of claim 14 wherein the Chlamydia protease is a Chlamydia tail specific protease.

18. The method of claim 14 wherein the Chlamydia protease is Chlamydia trachomatis tail-specific protease CT441, Chlamydia pneumoniae tail-specific protease Cpn0555, or Chlamydia muridarum tail-specific protease TC0725.

19. A method for identifying a compound capable of inhibiting Chlamydia infection of a cell comprising,
contacting a test compound with a cell-based assay system comprising a cell expressing Chlamydia protease and capable of signaling responsiveness to NF-κB, and
detecting an effect of the test compound on NF-κB activation in the presence of TNFα in the cell-based assay system as an increase or a decrease in susceptibility of the cell to Chlamydia infection, effectiveness of the test compound in the assay being indicative of the inhibition of Chlamydia infection of the cell.

20. The method of claim 19 wherein the test compound inhibits Chlamydia protease activity and restores NF-κB activation in the presence of TNFα indicating a decrease in susceptibility of the cell line to Chlamydia infection in the presence of the test compound.

21. The method of claim 19 wherein the test compound is a small chemical molecule, interfering RNA, dominant-negative molecule, short hairpin RNA, ribozyme, antisense oligonucleotide, protein inhibitor, monoclonal antibody, polyclonal antibody, peptide, peptidomimetic, or a nucleic acid.

22. The method of claim 19 which further comprises detecting a decrease in Chlamydia protease cleavage of p65/RelA to p40.

23. The method of claim 22 which further comprises detecting a decrease in susceptibility of the cell line to Chlamydia infection.

24. The method of claim 19 wherein the Chlamydia protease is a Chlamydia tail specific protease, Chlamydia trachomatis tail-specific protease CT441, Chlamydia pneumoniae tail-specific protease Cpn0555, or Chlamydia muridarum tail-specific protease TC0725.

25. The method of claim 19 wherein the cell based assay system comprises Hela 229 cells, 293T cells, or NIH3T3 cells.
